(19) United States
(12) Patent Application Publication
(10) Pub. No.: US 2023/0058271 A1
(43) Pub. Date: Feb. 23, 2023

(54) METHODS, SYSTEMS, AND DEVICES FOR PROCESSING HEALTH-RELATED INFORMATION

(71) Applicant: CXA GROUP PTE. LIMITED, Singapore (SG)

(72) Inventor: Rosaline Chow Koo, Singapore (SG)

(21) Appl. No.: 17/794,904

(22) PCT Filed: Jan. 22, 2020
(86) PCT No.: PCT/SG2020/050032

§ 371 Date: Jul. 22, 2022

(51) Int. Cl. G06Q 10/10; G16H 50/30

(52) U.S. Cl. CPC G06Q 10/1057; G16H 50/30

(57) ABSTRACT

Embodiments relate to a method for processing and/or managing health-related information. An example method may include establishing one or more communication channels, receiving a plurality of information, processing received information, deriving a first ranked list of recommendations, deriving a second ranked list of recommendations, deriving a third ranked list of recommendations and deriving a final ranked list of recommendations. The method for processing and/or managing health-related information may further include responsive to a selection of a recommendation by the user, updating the current set of information and the health score based on the selected recommendation. The method for processing and/or managing health-related information may further include generating a modified health score for each recommendation wherein the modified health score for each recommendation represents the health score for the user if the recommendation is selected.

### TECHNICAL FIELD

The present disclosure relates generally to processing of health-related information, and more specifically, to methods, systems, devices, and logic for receiving, processing, and managing health-related information.

### BACKGROUND

Employer sponsored healthcare benefits are important in terms of an employee's job satisfaction and most organizations offer medical insurance to their full time employees. These benefits constitute a significant portion of an organization's operating costs. Furthermore, healthcare and health insurance costs have been increasingly expensive globally and the increasing incidence of chronic diseases such as cardiovascular diseases, diabetes and cancer resulting in the organization bearing more for medical related products and services.

In addition to having to incur the inflating costs of medical related products and services, the employer may have to deal with large claims made by the employees due to ill health, indirect loss such as significantly reduced productivity due to absenteeism and presentism of employees who are ill and all of this translates to cost incurred exceeding the earnings of the employees or organization and inflation.

Studies show that early identification and management of chronic illnesses may be beneficial to both employees and employers. Early identification of diseases may help to identify complications of diseases and to manage it before they become severe. For example, early detection of high blood sugar levels may indicate that the employee is at risk of long term complications such as neuropathy, kidney failure and cardiovascular disease. It is also especially dangerous if the employee is diabetic. Emergencies may occur if left unmonitored and situations as such will result in costly hospitalization, medical services and medication. The cost of these expensive emergencies can be prevented by improving the management of the chronic disease.

### BRIEF SUMMARY

Accordingly, a need exists for a system that allows the focus to be shifted from treating an illness to early detection and preventive care at a personalized level for the employee and employer.

Present example embodiments relate generally to and/or include systems, subsystems, processors, devices, logic, methods, and processes for addressing conventional problems, including those described above and in the present disclosure, and more specifically, example embodiments relate to systems, subsystems, processors, devices, logic, methods, and processes for receiving, processing, and managing health-related information, or the like, for a plurality of users.

In an exemplary embodiment, a method is described. The method may be for processing a plurality of information by establishing one or more communication channels via a processor. The method may include receiving a plurality of information for a first user via the established communication channels. The plurality of users may be users of the platform (e.g. employees). The method may also include processing the received information for the first user by deriving a first ranked list for the first user. The first ranked list is derived by selecting one or more health-related information from the received information of the first user. The health-related information for the first user may include information pertaining to metabolic information of the first user (e.g. body mass index, glucose levels, triglyceride levels, high density lipoprotein cholesterol levels and blood pressure information). The method may include searching the recommendations database for available recommendations. The recommendation database may include a plurality of health-related recommendations. The selected health-related recommendations may be recommendations selected from a health perspective for the first user. The method may include establishing a first score for each of the selected health-related recommendations based on the selected one or more health-related information for the first user. The method may also include ranking the selected health-related recommendations to arrive at the first ranked list for the first user. The ranking of the selected health-related recommendations for the first user is based on the first score of each of the selected health-related recommendations for the first user. The method may also include processing the received information for the first user by deriving a second ranked list for the first user. The second ranked list is derived by selecting one or more financial-related information from the received information of the first user. The method may include searching the recommendations database for available recommendations. The recommendation database may also include a plurality of financial-related recommendations. The selected financial-related recommendations may be recommendations selected from a financial perspective of the first user. The method may include establishing a second score for each of the selected health-related recommendations based on the selected one or more financial -related information for the first user. The method may also include ranking the selected financial-related recommendations to arrive at the second ranked list for the first user. The ranking of the selected financial-related recommendations for the first user is based on the second score of each of the selected financial-related recommendations for the first user.

In another exemplary embodiment, a method is described. The method may be for processing a healthcare-related of information by receiving a current set of information for a first user. The plurality of users may be users of the platform (e.g. employees). The current set of information for the first user may include a set of metabolic information (e.g. body mass index, glucose level, triglyceride level, high density lipoprotein (LDL) cholesterol level, and blood pressure information) for the first user and a set of lifestyle information (e.g. health risk assessment (HRA) for the first user, number of steps by the first user, and images) for the first user. The method may include processing the received current set of information for the first user and transforming the current set of information for the first user to a health score for the first user. The method may also include transforming of the current set of information for the first user to the health score for the first user. The method may include transforming of the current set of information which includes transforming the set of metabolic information for the first user to a metabolic score for the first user. The method may also include transforming of the current set of information which includes transforming the set of lifestyle information for the first user to a lifestyle score for the first user. A health score is determined based on the metabolic score and lifestyle score. The method may include displaying the determined health score on a graphical display of the first user. The method may include generating a first set of recommendations based on the current set of information for the first user and displaying the first set of recommendations (products, medication, services, lifestyle changes and/or fitness) on the graphical display of the first user. The first user may make a selection of one of the recommendations from the first set of recommendations. The method may also include updating the current set of information for the first user based on the recommendation selected by the first user. The health score is updated based on the recommendation that was selected by the first user and the updated health score for the first user is displayed on the graphical display of the first user. The method may include generating a second set of recommendations based on the current set of information for the first user and displayed on the graphical display of the first user.

In another exemplary embodiment, a method is described. The method may be for processing a healthcare-related of information by receiving a current set of information for a first user. The plurality of users may be users of the platform (e.g. employees). The current set of information for the first user may include a set of metabolic information (e.g. body mass index, glucose level, triglyceride level, high density lipoprotein (LDL) cholesterol level, and blood pressure information) for the first user and a set of lifestyle information (e.g. health risk assessment (HRA) for the first user, number of steps by the first user, and images) for the first user. The method may include processing the received current set of information for the first user and transforming the current set of information for the first user to a health score for the first user. The method may also include transforming of the current set of information for the first user to the health score for the first user. The method may include transforming of the current set of information which includes transforming the set of metabolic information for the first user to a metabolic score for the first user. The method may also include transforming of the current set of information which includes transforming the set of lifestyle information for the first user to a lifestyle score for the first user. A health score is determined based on the metabolic score and lifestyle score. The method may include displaying the determined health score on a graphical display of the first user. The method may include generating a first set of recommendations based on the current set of information for the first user and displaying the first set of recommendations (products, medication, services, lifestyle changes and/or fitness) on the graphical display of the first user. The method may also include generating an achievable health score for each recommendation in the first set of recommendations including a first achievable health score for the first recommendation. The achievable health score for each recommendation in the first set of recommendations represent what the health score will or may be if the first user selects the said recommendation. The first achievable health score represents the health score for the first user if the first recommendation is selected. The achievable health score for each recommendation in the first set of recommendations, including the first achievable health score for the first recommendation, is displayed on the graphical display of the first user.

### BRIEF DESCRIPTION OF THE DRAWINGS

For a more complete understanding of the present disclosure, example embodiments, and their advantages, reference is now made to the following description taken in conjunction with the accompanying drawings, in which like reference numbers indicate like features, and:

FIG. 1A is an illustration of an example system for processing claims;

FIG. 1B is an illustration of an example method for processing claims;

FIG. 2 is an illustration of an example embodiment of a system for processing claims;

FIG. 3 is an illustration of an example embodiment of a system for processing and/or managing health-related information;

FIG. 4 is an illustration of an example embodiment of a processor for processing and/or managing healthcare-related information;

FIG. 5 is an illustration of an example embodiment of a health processor or health engine;

FIG. 6 is an illustration of an example embodiment of a financial processor or financial engine;

FIG. 7 is an illustration of an example embodiment of a prediction processor or prediction engine;

FIG. 8 is an illustration of an example embodiment of a final recommendation processor or final recommendation engine;

FIG. 9 is an illustration of an example embodiment of a health score processor or health score engine;

FIG. 10 is an illustration of an example embodiment of a method of deriving a final ranked list of recommendations based on a first ranked list, second ranked list, and/or third ranked list of recommendations;

FIG. 10A is an illustration of an example embodiment of deriving a first ranked list of recommendations;

FIG. 10B is an illustration of an example embodiment of deriving a second ranked list of recommendations;

FIG. 10C is an illustration of an example embodiment of deriving a third ranked list of recommendations;

FIG. 10D is an illustration of an example embodiment of deriving a final ranked list of recommendations;

FIG. 11 is an illustration of an example embodiment of a method of processing health scores for a user; and

FIG. 12 is an illustration of an example embodiment of a method of generating achievable health scores.

Although similar reference numbers may be used to refer to similar elements in the figures for convenience, it can be appreciated that each of the various example embodiments may be considered to be distinct variations.

Example embodiments will now be described with reference to the accompanying drawings, which form a part of the present disclosure and which illustrate example embodiments which may be practiced. As used in the present disclosure and the appended claims, the terms “embodiment,” “example embodiment,” “exemplary embodiment,” and “present embodiment” do not necessarily refer to a single embodiment, although they may, and various example embodiments may be readily combined and/or interchanged without departing from the scope or spirit of example embodiments. Furthermore, the terminology as used in the present disclosure and the appended claims is for the purpose of describing example embodiments only and is not intended to be limitations. In this respect, as used in the present disclosure and the appended claims, the term “in” may include “in” and “on,” and the terms “a,” “an,” and “the” may include singular and plural references. Furthermore, as used in the present disclosure and the appended claims, the term “by” may also mean “from,” depending on the context. Furthermore, as used in the present disclosure and the appended claims, the term “if” may also mean “when” or “upon,” depending on the context. Furthermore, as used in the present disclosure and the appended claims, the words “and/or” may refer to and encompass any and all possible combinations of one or more of the associated listed items.

### DETAILED DESCRIPTION

Presently, when a user visits a service provider (e.g., when an employee visits a medical clinic, hospital, rehabilitation center, etc. and/or a virtual service provider (e.g., virtual doctors, tele-docs, virtual medical centers, tele-meds, etc.) for a medical treatment or a medical check up), fees for services, products, medication, etc. provided by, during and/or pursuant to the visit may be payable or paid, in part or in full, by the user, an insurance company (e.g., via a third party such as a third party administrator (TPA), broker, representative, or the like, of or for the insurance company), etc. For payments and/or reimbursements of such fees, directly or indirectly, by an insurance company (e.g., via the third party such as a third party administrator (TPA), broker, representative, or the like, of or for the insurance company), the user and/or the service provider is/are typically required to submit a claim (hereinafter referred to as claims, medical claims, or the like) to the insurance company (e.g., via the third party such as a third party administrator (TPA), broker, representative, or the like, of or for the insurance company) for the visit. In submitting each claim, the service provider is typically required to manually enter all relevant/required information from the visit into separate computer systems (e.g., a clinical management system (or CMS) of or for the service provider, a system of or for a third party administrator (TPA), broker, representative, or the like, of or for the insurance company, etc.). The submitted claim is then processed (e.g., by the third party such as a third party administrator (TPA), broker, representative, or the like, of or for the insurance company) to determine, among other things, whether the user's insurance policy is valid; whether there may be suspicious and/or fraudulent activities; whether all of the required information and/or documentation have been provided by the service provider and/or user as part of the submitted claim; whether the service provider is an eligible service provider under the user's insurance policy; whether the services, products, medication, etc. is/are eligible to be covered by the user's insurance policy; etc. In situations when the processing is successful, the insurance company is notified of such processed claims, and payment is made to the service provider (and/or reimbursements made to the user for payments already paid by the user to the service provider). While such a process and system has been widely implemented and used in many parts of the world, it is recognized in the present disclosure that certain problems and/or inefficiencies may be encountered with such processes and systems.

For example, in certain countries and/or regions, the processing of claims for services, products, medication, etc. provided by, during, and/or pursuant to a visit to a service provider may require a multitude of steps and/or actions, as described above and in the present disclosure, performed by, for, and/or on behalf of one or more entities including, but not limited to, the user himself/herself (e.g., employee, customer, client, subscriber, etc.), the service provider (e.g., medical clinic, hospital, rehabilitation center, etc.), an insurance company providing insurance coverage to the user (e.g., via one or more third parties such as third party administrators (TPA), brokers, aggregators, coordinators, and/or representatives), and/or an entity who is responsible for payment of the user's medical insurance policy (e.g., employer, financial institution, travel company, etc.).

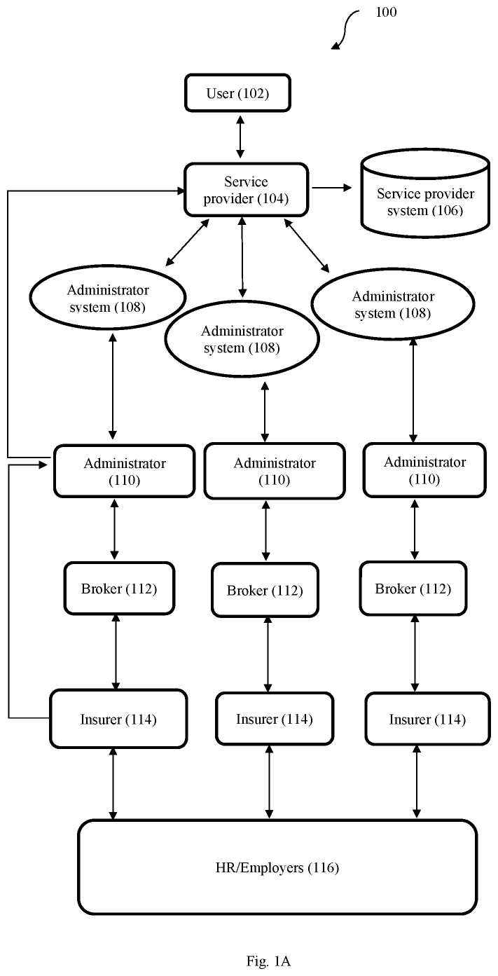

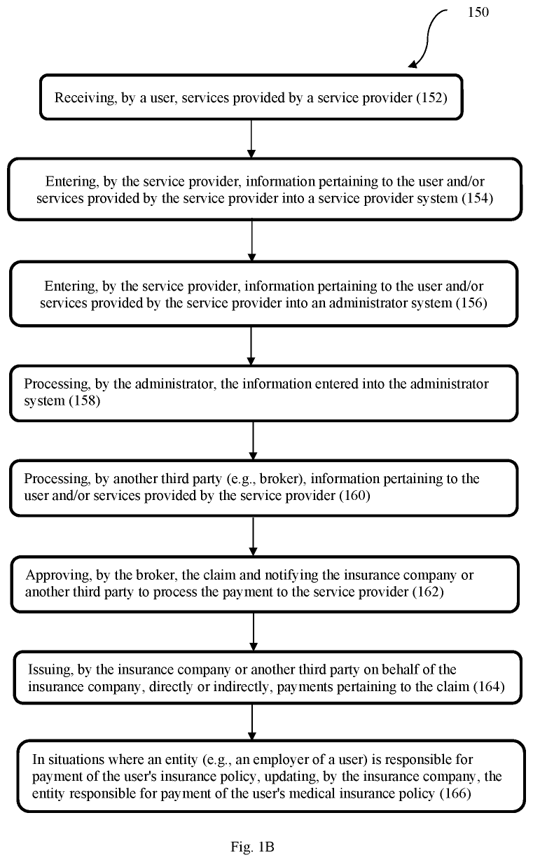

FIG. 1A illustrates an example of a system or network 100 for processing claims in certain countries and/or regions. As illustrated in FIG. 1A, when a user (e.g., user 102, which may include an employee, customer, client, subscriber, etc.) visits a service provider (e.g., service provider 104, which may include a medical clinic, hospital, rehabilitation center, etc. and/or a virtual service provider (e.g., virtual doctors, tele-docs, virtual medical centers, tele-meds, etc.) and is provided with a service, product, medication, etc. (e.g., medical treatment, check up, prescription drugs, etc. that may be covered by an insurance policy), the service provider 104 may be required to manually enter into several computer systems information pertaining to the user, the service provider, and/or services, products, medication, etc. provided by, during, and/or pursuant to the visit. For example, the service provider may be required to enter some or all such information into a service provider computer system (e.g., service provider system 106, which may include a clinical management system (CMS) or other system owned, controlled, managed, assigned, subscribed, and/or assigned by or for the service provider 104). As another example, the service provider 104 may be required to enter some or all such information into a computer system of or for a third party computer system (e.g., administrator system 108). Such administrator systems 108 may be owned, controlled, managed, assigned, subscribed, and/or accessible by or for third parties (e.g., administrators 110, which may include third party administrators (TPA), governmental entities or agencies, etc.). It is recognized in the present disclosure that such information required to be entered into the administrator systems 108 may include some or all of the same or similar information that is required to be entered into the service provider systems 106, and vice versa.

Upon accessing the information from the administrator system 108, the administrators 110 may process the information. The processing may include, among other things, ensuring the user's insurance policy is valid; ensuring there are no suspicious and/or fraudulent activities associated with the claim; ensuring all of the required information and/or documentation have been received by the service provider and/or user as part of the submitted claim; ensuring the service provider is an eligible service provider under the user's insurance policy; ensuring the services, products, medication, etc. is/are eligible to be covered by the user's insurance policy; etc. The administrators 110 may notify the insurance company (e.g., insurance company 114) of the results of such processed claims, and/or may send information to one or more other third parties 112 (e.g., brokers). Such brokers 112 may be responsible for reviewing the information and/or processing from the administrators 110; performing further processing (e.g., ensuring the user's insurance policy is valid; ensuring there are no suspicious and/or fraudulent activities associated with the claim; ensuring all of the required information and/or documentation have been received by the service provider and/or user as part of the submitted claim; ensuring the service provider is an eligible service provider under the user's insurance policy; ensuring the services, products, medication, etc. is/are eligible to be covered by the user's insurance policy; etc.); approving (or rejecting) the claims; and/or notifying the insurance company 114 of the results of the processing. For approved claims, the brokers 112 may process payment to the service providers so as to be paid by the brokers 112 themselves, by the insurance company 114, and/or via the administrators 110. In situations when a separate entity (e.g., an employer 116 of the user 102) is responsible for payment of the user's medical insurance policy, the insurance company 114 may also inform the entity 116 of the details of the user's medical claims. It is recognized in the present disclosure that in processing and settling such claims, the insurance company 114 will pay the service provider 104 and also the administrators 110 and brokers 112 for their services rendered, as described above and in the present disclosure.

FIG. 1B illustrates an example method (e.g., method 150) of processing claims using the system or network 100. The method 150 may include receiving, by a user, one or more services, products, medications, etc. provided by, during, and/or pursuant to a visit to a service provider (e.g., action 152). The method 150 may include entering, by the service provider into a service provider system, information pertaining to the service, product, medication, etc. provided by, during, and/or pursuant to the visit to the service provider (e.g., action 154). Such submitted claim may include information pertaining to the user, the service provider, and/or service, product, medication, etc. provided by, during, and/or pursuant to the visit to the service provider. For example, information may include the user's medical details and medical history, details of user's visit to the service provider including services and/or products provided to the user, medical prescriptions issued to the user, costs, etc. The service provider may be required to submit a claim for the visit into one or more administrator systems (e.g., systems of third party administrators (TPA), governmental entities or agencies, etc.) (e.g., action 156). The claim may include some or all of the same or similar information entered into the service provider system pertaining to the services, products, medications, etc. and/or the user. For example, the information may include information about the service provider, information on the user's insurance policy, user's medical details and medical history, details of user's visit to the service provider including services and/or products provided to the user, medical prescriptions issued to the user, costs, etc. The method 150 may further include processing, by the administrator, the information entered into the administrator system (e.g., action 158). The processing may include, among other things, ensuring the user's insurance policy is valid; ensuring there are no suspicious and/or fraudulent activities associated with the claim; ensuring all of the required information and/or documentation have been received by the service provider and/or user as part of the submitted claim; ensuring the service provider is an eligible service provider under the user's insurance policy; ensuring the services, products, medication, etc. is/are eligible to be covered by the user's insurance policy; etc. Such processing may be performed to ensure the claims are not fraudulent, not missing information, and/or comply and adhere to specific rules, fee schedules, forms, etc. The method 150 may further include processing, by another third party (e.g., a broker) some or all of the same or similar information processed by the administrator (e.g., action 160). The processing by the broker may include some or all of the actions performed (or not performed) by the administrator, including, but not limited to, ensuring the user's insurance policy is valid; ensuring there are no suspicious and/or fraudulent activities associated with the claim; ensuring all of the required information and/or documentation have been received by the service provider and/or user as part of the submitted claim; ensuring the service provider is an eligible service provider under the user's insurance policy; ensuring the services, products, medication, etc. is/are eligible to be covered by the user's insurance policy; etc. The method 150 may further include approving, by the broker (or by the insurance company via the broker), the claim and notifying the insurance company or another third party to process payment to the service provider (e.g., action 162). The method 150 may further include issuing, by the insurance company or another third party on behalf of the insurance company, directly or indirectly, payments pertaining to the claim (e.g., action 164). Such payments may include, but are not limited to, payments to the service provider; payments to the broker; payments to the administrator; and/or payments to the user (e.g., as reimbursements for payments already made to the service provider). In situations where the insurance company does not make payments directly to the service provider, the insurance may make payments to the administrator and/or broker, and the administrator and/or broker would then make payments to the service provider. In situations where an entity (e.g., an employer of a user) is responsible for payment of the user's medical insurance policy, the method 150 may include updating, by the insurance company, the entity responsible for payment of the user's medical insurance policy (e.g., action 166). It is recognized in the present disclosure that such a method 150 and system 100 requires a multitude of actions or steps by multiple entities, and corresponding payments to such multiple entities. For example, it is estimated that between about 15-20% (or more or less) of the total amount of each claim payment paid by the insurance company represents the service fee to the broker and another between about 15-20% of the total amount of each claim payment paid by the insurance company represents the service fee to the administrator. As a result, insurance premiums paid for a user's medical insurance policy will need to cover such service fee payments to the brokers and administrators. It is recognized in the present disclosure that insurance premiums paid for insurance policies and amounts paid by insurance companies for settling claims can be reduced by example embodiments of methods and systems described in the present disclosure.

As another example of problems encountered today, most employers tend to manage their expenditure in healthcare for their employees after a large amount of expenses or claims have been made by their employees. In order to manage the inflating costs, the employers tend to shift the healthcare expenses to the employees themselves. This approach requires employees to pay a greater share of healthcare costs. Alternatively, the employers may revise their healthcare coverage for their employees in order to reduce the inflating costs of healthcare. At times, the revisions do not take into consideration the current health status and to a certain extent, the current financial status of the employee. Revisions may also not take into consideration life work events (LWE) that have or are occurring in the personal or work aspect of the employee's life. Some examples of personal-based life work events may include the employee getting married or divorced, having or adopting a baby, or coping with a death in the family etc. Some examples of work-based life work events may include events such as joining or leaving of a company, getting a promotion or demotion, changes in income due to wage increments or bonuses etc. The revisions may therefore result in insufficient health coverage that may eventually lead to additional costs to be incurred and this method does not reduce the expenses for healthcare for both parties.

Conventional solutions for controlling costs have provided employers and employees with programs or systems geared towards managing and providing health care for employees. However, such solutions generally do not take into account information, measurements and/or statistics of the current health status of employees, and therefore such systems are inherently unable to find appropriate solutions to solve specific needs of the employee and the employer. Specifically, existing programs and systems are typically a one-size-fits-all programs which will not be beneficial or helpful to an employee who may have specific or existing needs (e.g., specific or existing medical conditions or diseases). In addition, some of these programs or systems may be developed without scientific basis and may not be able to provide an accurate or complete assessment. Consequently, these available methods do not assist in a meaningful or personalized manner and do not achieve the aim to reduce healthcare costs.

Accordingly, a need exists for a system that allows for improved processing of claims, reducing insurance premiums paid for insurance policies, reducing amounts paid by insurance companies for settling claims, and shifting of focus from treatment (e.g., treating an illness) to prevention (e.g., early detection and preventive care at a personalized level for the employee and employer).

Example Embodiments of a System for Processing Claims (e.g., System 200).

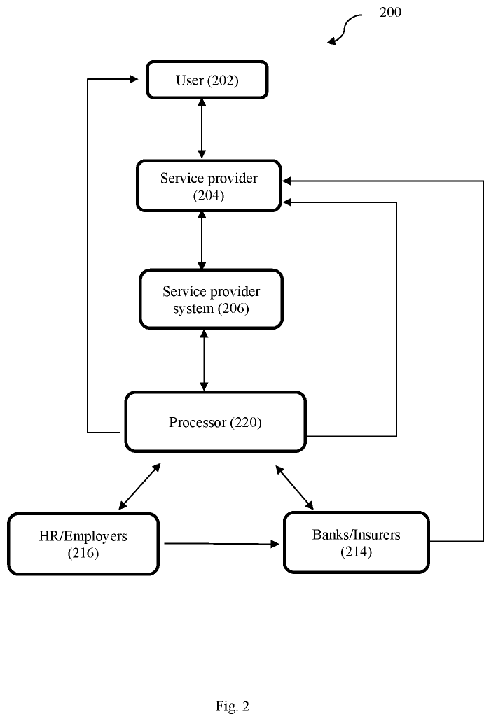

FIG. 2A illustrates an example embodiment of a system (e.g., system 200) for processing and/or managing claims. As illustrated in FIG. 2A, a user (e.g., user 202, which may include an employee, customer, client, subscriber, etc.) may visit a service provider (e.g., service provider 204, which may include a medical clinic, hospital, rehabilitation center, etc.). A service, product, medication, etc. (e.g., medical treatment, check up, prescription drugs, etc. that may be covered by an insurance policy) may be provided by, during, and/or pursuant to the visit to the service provider. To store information on each visit, the service provider 204 may enter information pertaining to the user, the service provider, and/or services, products, medication, etc. provided by, during, and/or pursuant to the visit into a service provider computer system (e.g., service provider system 106, which may include a clinical management system (CMS) or other system owned, controlled, managed, assigned, subscribed, and/or assigned by or for the service provider 104). As an improvement over the embodiments described above and illustrated in FIGS. 1A and 1B, example embodiments of the system 200 do not require the service provider 204 to manually enter into other computer systems (e.g., the administrator system 108 illustrated in FIG. 1A or any other third party computer systems) some or all of the same or similar information pertaining to the user, the service provider, the services, products, medication, etc. provided by, during, and/or pursuant to the visit to the service provider, etc.

Example embodiments of the system 200 may include one or more processors (e.g., processor 220). An example embodiment of the processor 220 may be configurable or configured to communicate with one or more computer systems or databases (e.g., the service provider system 206) so as to receive and/or retrieve information pertaining to a user's visit to a service provider. The processor 220 may also receive and/or retrieve information from the user (e.g., via a mobile app on a device of the user). Such received or retrieved information from the service provider system 206 and/or the user's device may include one or more of the following: information about the service provider; information on the user's insurance policy; information on the user's medical details and medical history; details of user's visit to the service provider including services and/or products provided to the user, medical prescriptions issued to the user, costs, etc.; other information pertaining to the user, such as biometrics, user activities (e.g., steps, distance travelled, heart rate, etc.), etc. An example embodiment of the processor 220 may be further configurable or configured to process the information. Such processing by the processor 220 may include ensuring the user's insurance policy is valid; ensuring there are no suspicious and/or fraudulent activities associated with the claim; ensuring all of the required information and/or documentation have been received by the service provider and/or user as part of the submitted claim; ensuring the service provider is an eligible service provider under the user's insurance policy; ensuring the services, products, medication, etc. is/are eligible to be covered by the user's insurance policy; etc. Such processing may include some or all of the processing, actions, checks, and/or verifications performed by administrators and/or brokers, as described above. In this regard, once the processor 220 processes the information, the processor 220 may be configurable or configured to process a claim for the visit to the service provider. The insurance company 214 may release payments directly to the service provider 204 (e.g., upon successful processing of the claim by the processor 220). In situations where an entity 216 (e.g., employer of the user) is responsible for payment of the user's insurance policy, the processor 220 may be configurable or configured to notify such entity 216 of the user's medical claims. Based on the information received or retrievable by the processor 220 as well as the processing performed by the processor 220, the processor 220 may be configurable or configured to manage, process, and/or provide health-related or health-based recommendations to the user, as further described in the present disclosure.

Example Embodiments of a System for Processing and/or Managing Health-Related Information (e.g., System 300).

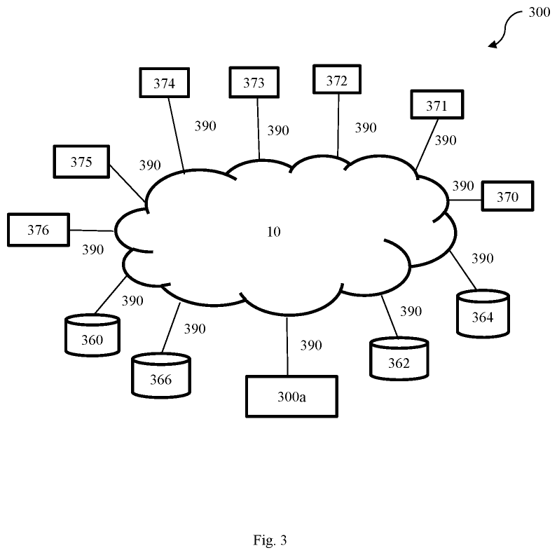

FIG. 3 illustrates an example embodiment of a system (e.g., system 300) for receiving, processing, and/or managing health-related information for a plurality of different types of users. Users of the system 300 may include, but are not limited to, employers (e.g., companies, government bodies, other organizations, etc.), employees, individuals, insurance companies, medical providers, wellness providers, retailers, other providers, etc.

As used in the present disclosure, when applicable, a reference to a system, subsystem, processor, sub-processor, engine, or the like, may also refer to, apply to, and/or include a computing device, processor, sub-processor, server, system, subsystem, cloud-based computing, or the like, and/or functionality of a processor, sub-processor, computing device, server, system, subsystem, cloud-based computing, or the like. The system 300 (and/or its elements, as described in the present disclosure) may be any processor, server, system, device, computing device, controller, microprocessor, microcontroller, microchip, semiconductor device, or the like, configurable or configured to perform, among other things, a processing and/or managing of information, data and/or voice communications, and/or other actions described above and in the present disclosure. Alternatively or in addition, the system 300 (and/or its elements, as described in the present disclosure) may include and/or be a part of a virtual machine, processor, computer, node, instance, host, or machine, including those in a networked computing environment. As used in the present disclosure, such a network and/or cloud may be a collection of devices connected by communication channels that facilitate communications between devices and allow for devices to share resources. Such resources may encompass any types of resources for running instances including hardware (such as servers, clients, mainframe computers, networks, network storage, data sources, memory, central processing unit time, scientific instruments, and other computing devices), as well as software, software licenses, available network services, and other non-hardware resources, or a combination thereof. A network or cloud may include, but is not limited to, computing grid systems, peer to peer systems, mesh-type systems, distributed computing environments, cloud computing environment, etc. Such network or cloud may include hardware and software infrastructures configured to form a virtual organization comprised of multiple resources which may be in geographically disperse locations. Network may also refer to a communication medium between processes on the same device. Also as referred to herein, a network element, node, or server may be a device deployed to execute a program operating as a socket listener and may include software instances.

An example embodiment of the system 300 may include a main processor (e.g., main processor 300a). The main processor 300a may be or include the processor 220, as described in the present disclosure. The main processor 300a may include one or more processors, sub-processors, or the like, or be configurable or configured to perform the functions, operations, controls, commands, or the like, of one or more processors, sub-processors, or the like. For example, the main processor 300a may include a health-related or health-based processor or engine (e.g., health engine 310). Alternatively or in addition, the main processor 300a may be configurable or configured to perform the functions of a health engine 310. As will be further described in the present disclosure, an example embodiment of the health engine 310 may be for use in selecting health-related or health-based information for one or more users; selecting health-related or health-based recommendations for each of the users (e.g., based on availability for the user, based on the selected health-related information for the user, etc.); scoring one or more of the selected health-related or health-based recommendations for each of the users (e.g., based on availability for the user, based on the selected health-related information for the user, etc.); and/or ranking one or more of the selected health-related or health-based recommendations for each of the users (e.g., based on the selected health-related information for the user, based on the scoring of the selected health-related or health-based recommendations, etc.). The health engine 310 may also be configurable or configured to derive a first ranked list for each of the users (e.g., a ranked list from a health perspective). It is to be understood in the present disclosure that the above processes and/or actions by the health engine 310 may be performed in the above order or different order without departing from the teachings of the present disclosure.

As another example, the main processor 300a may include a financial-related or financial-based processor or engine (e.g., financial engine 320). Alternatively or in addition, the main processor 300a may be configurable or configured to perform the functions of a financial engine 320. As will be further described in the present disclosure, an example embodiment of the financial engine 320 may be for use in selecting financial-related or financial-based information for one or more users; selecting financial-related or financial-based recommendations for each of the users (e.g., based on availability for the user, based on the selected financial-related information for the user, etc.); scoring one or more of the selected financial-related or financial-based recommendations for each of the users (e.g., based on availability for the user, based on the selected financial-related information for the user, etc.); and/or ranking one or more of the selected financial-related or financial-based recommendations for each of the users (e.g., based on the selected financial-related information for the user, based on the scoring of the selected financial-related or financial-based recommendations, etc.). The financial engine 320 may also be configurable or configured to derive a second ranked list for each of the users (e.g., a ranked list from a financial perspective). It is to be understood in the present disclosure that the above processes and/or actions by the financial engine 320 may be performed in any order without departing from the teachings of the present disclosure.

As another example, the main processor 300a may include a prediction-related or prediction-based processor or engine (e.g., prediction engine 330). Alternatively or in addition, the main processor 300a may be configurable or configured to perform the functions of a prediction engine 330. As will be further described in the present disclosure, an example embodiment of the prediction engine 330 may be for use in selecting historical and/or prediction-based information for one or more users (e.g., historic recommendations accepted or selected by the user, historic recommendations not accepted or not selected by the user, historic recommendations not recommended to the user, etc.); selecting prediction-related or prediction-based recommendations for each of the users (e.g., based on availability for the user, based on the selected historical and/or prediction-based information for the user, etc.); scoring one or more of the selected prediction-related or prediction-based recommendations for each of the users (e.g., based on availability for the user, based on the selected historical and/or prediction-based information for the user, etc.); and/or ranking one or more of the selected prediction-related or prediction-based recommendations for each of the users (e.g., based on the selected historical and/or prediction-related information for the user, based on the scoring of the selected historical and/or prediction-related or prediction-based recommendations, etc.). The prediction engine 330 may also be configurable or configured to derive a third ranked list for each of the users (e.g., a ranked list from a historical and/or predictive perspective). It is to be understood in the present disclosure that the above processes and/or actions by the prediction engine 330 may be performed in any order without departing from the teachings of the present disclosure.

As another example, the main processor 300a may include a final recommendation processor or engine (e.g., final recommendation engine 340). Alternatively or in addition, the main processor 300a may be configurable or configured to perform the functions of a final recommendation engine 340. As will be further described in the present disclosure, an example embodiment of the final recommendation engine 340 may be for use in processing the first ranked list, the second ranked list, and/or the third ranked list based on one or more predetermined criterion (e.g., equally or unequally weighted, etc.). The final recommendation engine 340 may also be configurable or configured to derive a final ranked list for each of the users. In an example embodiment, such final ranked list is or includes a ranked list of recommendations for each of the users.

As another example, the main processor 300a may include a health score processor or engine (e.g., health score engine 350). Alternatively or in addition, the main processor 300a may be configurable or configured to perform the functions of a health score engine 350. As will be further described in the present disclosure, an example embodiment of the health score engine 350 may be for use in transforming information for each user to a health score for the user. In an example embodiment, the health score for each user may include a metabolic score and/or a lifestyle score.

The system 300 may also include a transmission processor or engine (e.g., transmission processor 380). The system 300 may also include one or more databases, or the like. For example, the system 300 may include a recommendations database (e.g., recommendations database 360). The system 300 may also include a historical selections database (e.g., historical selections database 362). The system 300 may also include one or more other databases (e.g., other databases 364).

The system 300 may also include and/or be configurable or configured to communicate with one or more employees (e.g., employees 370). As used in the present disclosure and represented in the figures, an employee 370 may include and/or represent employee portable computing devices, employee wearable devices, employee computer systems, employee cloud computing, employee databases, etc.). For example, the employee 370 may include and/or represent a wearable computing device which may take the form of a personal devices (e.g., laptop, desktop computer, mobile phones, etc.), a device mounted on the user's body or embedded in clothing (e.g., smartwatch, fitness tracker, sensors, etc.), computerized medical devices (e.g., electronic blood sugar monitor, electronic blood pressure monitor, etc.). The system 300 may also include and/or be configurable or configured to communicate with one or more employers (e.g., employers 371). As used in the present disclosure and represented in the figures, an employer 371 may include and/or represent employer portable computing devices, employer computer systems, employer cloud computing, employer databases, human resources databases, employer-accessible government or third-party databases, etc.). The system 300 may also include and/or be configurable or configured to communicate with one or more insurance companies (e.g., insurance companies 372). As used in the present disclosure and represented in the figures, an insurance company 372 may include and/or represent insurance company portable computing devices, insurance company computer systems, insurance company cloud computing, insurance company databases, insurance company-accessible government or third-party databases, etc.). The system 300 may also include and/or be configurable or configured to communicate with one or more medical provider (e.g., medical provider 373). As used in the present disclosure and represented in the figures, a medical provider 373 may include and/or represent medical provider portable computing devices, medical provider computer systems, medical provider cloud computing, medical provider databases, medical provider-accessible government or third-party databases, etc.). The system 300 may also include and/or be configurable or configured to communicate with one or more wellness provider (e.g., wellness provider 374). As used in the present disclosure and represented in the figures, a wellness provider 374 may include and/or represent wellness provider portable computing devices, wellness provider computer systems, wellness provider cloud computing, wellness provider databases, wellness provider-accessible government or third-party databases, etc.). The system 300 may also include and/or be configurable or configured to communicate with one or more retailers (e.g., retailers 375). As used in the present disclosure and represented in the figures, a retailer 375 may include and/or represent retailer portable computing devices, retailer computer systems, retailer cloud computing, retailer databases, retailer-accessible government or third-party databases, etc.). The system 300 may also include and/or be configurable or configured to communicate with one or more other providers (e.g., other provider 376). As used in the present disclosure and represented in the figures, an other provider 376 may include and/or represent other provider portable computing devices, other provider computer systems, other provider cloud computing, other provider databases, other provider-accessible government or third-party databases, etc.). The system 300 may also include and/or be configurable or configured to communicate via one or more wired and/or wireless communication channels (e.g., communication channel 390).

Example embodiments of the system 300 may include, not include, communicate with, and/or not communicate with one or more of the elements described above and in the present disclosure, may include and/or communicate with additional elements, may include and/or communicate with equivalent elements, may be formed, implementable/implemented, and/or used in different sequences, actions, combinations, and/or configurations, and/or one or more of the elements (and/or elements of elements) may be combinable into a single element and/or divided into two or more elements. It is also to be understood in the present disclosure that one or more functionalities, actions, methods, and/or processes performed or performable by one or more of the elements described above and in the present disclosure may be implemented or implementable, directly or indirectly, using or via one or more other elements or ways, such as via cloud computing, parallel, collaborative, and/or distributed computing or processing, distributed ledger technology (DLT), artificial intelligence (AI), machine learning, or the like. Communication using technologies other than the Internet are also contemplated in example embodiments without departing from the teachings of the present disclosure. The system 300, and elements and functionality thereof, will now be further described with reference to the accompanying figures and actions and methods described in the present disclosure.

Main Processor (e.g., Main Processor 300a)

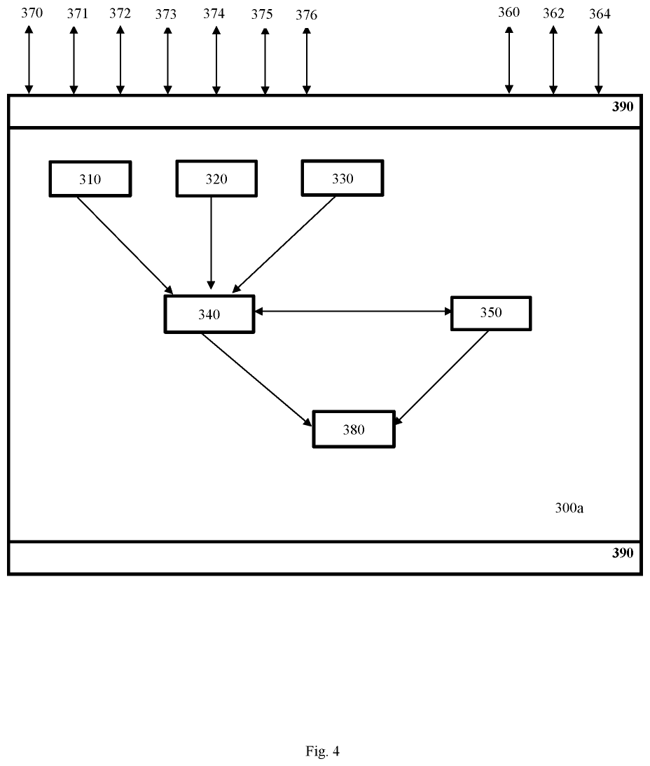

As illustrated in at least FIG. 3 and FIG. 4, an example embodiment of the system 300 may include a main processor (e.g., main processor 300a). The main processor 300a may include one or more processors, elements, sub-processors, virtual machines, or the like, for performing one, some, or all of the actions, functions, processing, processes, procedures, sub-processing, sub-processes, sub-procedures, or the like, disclosed in the present disclosure. Alternatively or in addition, the main processor 300a may be configurable or configured to perform one, some, or all of the actions, functions, processing, processes, procedures, sub-processing, sub-processes, sub-procedures, or the like, disclosed in the present disclosure.

The main processor 300a may include a communication or transmission processor (e.g., communication processor 380). Alternatively or in addition, the main processor 300a may be configurable or configured to perform the functions of a communication processor 380. The main processor 300a may also include a health-related or health-based processor or engine (e.g., health engine 310). Alternatively or in addition, the main processor 300a may be configurable or configured to perform the functions of a health engine 310. The main processor 300a may also include a financial-related or financial-based processor or engine (e.g., financial engine 320). Alternatively or in addition, the main processor 300a may be configurable or configured to perform the functions of a financial engine 320. The main processor 300a may also include a prediction-related or prediction-based processor or engine (e.g., prediction engine 330). Alternatively or in addition, the main processor 300a may be configurable or configured to perform the functions of a prediction engine 330. The main processor 300a may also include a final recommendation processor or engine (e.g., final recommendation engine 340). Alternatively or in addition, the main processor 300a may be configurable or configured to perform the functions of a final recommendation engine 340. The main processor 300a may also include a health score processor or engine (e.g., health score engine 350). Alternatively or in addition, the main processor 300a may be configurable or configured to perform the functions of a health score engine 350. These and other elements of the main processor 300a are further described below and in the present disclosure.

The Communication Processor (e.g., Communication Processor 380).

As illustrated in at least FIG. 4, the main processor 300a may include a communication processor 380 (e.g., communication processor 380). The communication processor 380 may be configurable or configured to establish one or more communication channels for receiving and/or transmitting information. For example, the communication processor 380 may be configurable or configured to receive and/or transmit information for one or more users. The communication processor 380 may do so by establishing one or more communication channels with one or more employees 370 to receive information for (or pertaining to or about) and/or from such employees 370 (e.g., employees as users). Alternatively or in addition, the communication processor 380 may establish one or more communication channels with one or more employees 370 to transmit information for (or pertaining to or about) and/or to such employees (e.g., recommendations to the employees as users, as will be further described in the present disclosure). The communication processor 380 may also establish one or more communication channels with one or more employers 370 of users to receive information for (or pertaining to or about) the users. The communication processor 380 may also establish one or more communication channels with one or more insurance companies 372 of users to receive information for (or pertaining to or about) the users. The communication processor 380 may also establish one or more communication channels with one or more medical providers 373 of users to receive information for (or pertaining to or about) the users. The communication processor 380 may also establish one or more communication channels with one or more wellness providers 374 of users to receive information for (or pertaining to or about) the users. The communication processor 380 may also establish one or more communication channels with one or more retailers 375 (e.g., to receive information from retailers having information for, pertaining to, or about the user). The communication processor 380 may also establish one or more communication channels with one or more other providers 376 to receive information for (or pertaining to or about) the users. It is to be understood in the present disclosure that the communication processor 380 may establish communication channels with one or more of the information sources described above and in the present disclosure so as to obtain/receive/request life work events (LWE) for one or more users. As used in the present disclosure, a life work event (LWE) broadly refers to a change in situation of a user. A life work event (LWE) may include, but is not limited to, personal life-related changes (life events) such as marriage, divorce, having a baby, adopting a child, a death in the family, illness or injury to the user, family member, friend, etc. A life work event (LWE) may also include, but is not limited to, work or employment-related changes (work events) such as a new employment, a change in employment, loss of employment, changes to income/remuneration (e.g., changes that can result in increment or decrement that affects insurance coverage that the user qualifies for), changes to employment category (e.g., changes that can affect coverage the user qualifies for), etc. In example embodiments, a life work event (LWE) may result in a user becoming eligible for a special enrollment period, allowing the user to enroll in group insurance outside a yearly open enrollment period, etc.

The Health Engine (e.g., Health Engine 310).

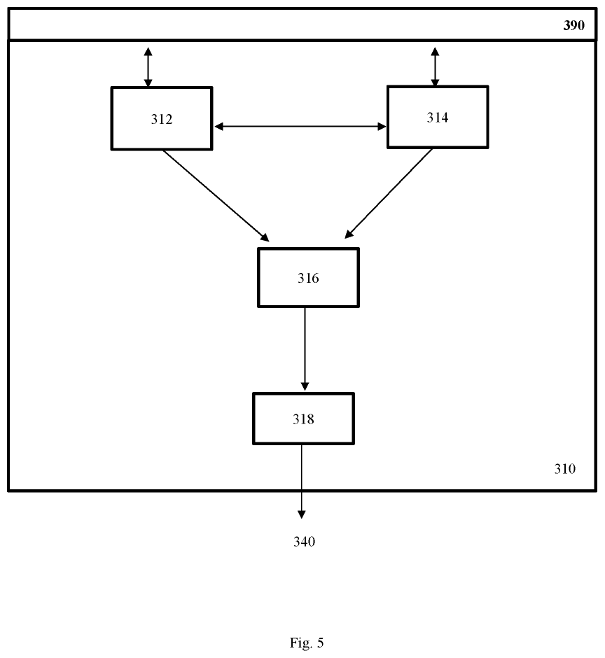

As illustrated in at least FIG. 4 and FIG. 5, the main processor 300a may include a health engine (e.g., health engine 310). Alternatively or in addition, the main processor 300a may be configurable or configured to perform the functions, processes, and/or actions of a health engine 310.

The health engine 310 may be configurable or configured to derive a first ranked list for one or more users. As will be further described in the present disclosure, the first ranked list derived for each user may then be used by, among other elements of the main processor 300a, the final recommendation engine 340 to arrive at a final ranked list of recommendations for each user.

The health engine 310 may include a health information selection engine (e.g., health information selection engine 312). Alternatively or in addition, the health engine 310 may be configurable or configured to perform the functions, processes, and/or actions of a health information selection engine 312. The health information selection engine 312 may be configurable or configured to select health-related information for each user. Such information selected may be from among the information received for (or pertaining to or about) the user (e.g., information obtained/received/requested by the communication processor 380, as described above and in the present disclosure).

The health engine 310 may also include a health recommendation selection engine (e.g., health recommendation selection engine 314). Alternatively or in addition, the health engine 310 may be configurable or configured to perform the functions, processes, and/or actions of a health recommendation selection engine 314. The health recommendation selection engine 314 may be configurable or configured to search a recommendations database 360. The recommendations database 360 may be or include a database of recommendations. For example, the recommendations database 360 may be or include a database of available recommendations. As another example, the recommendations database 360 may be or include a database of available recommendations based on a predetermined criterion (e.g., recommendations within a geographical location of the user, such as radius, district, city, province/state, country, etc.; available time period of the recommendation; recommendations available for the user based on employment, such as position, employer, etc.; recommendations available for the user based on insurance, such as insurance policy, insurance company, etc.; etc.).

The health recommendation selection engine 314 may also be configurable or configured to select, based on the searching of the recommendations database 360, health-related recommendations for each user. The health-related recommendations for each user may be selected based on a predetermined criterion. For example, the health-related recommendation for each user may be selected based on, among other things, availability, geographical location, etc. of the recommendation. As another example, the health-related recommendation for each user may be selected from a health perspective. As another example, the health-related recommendation for each user may be selected based on, among other things, the health-related information selected for each user by the health engine 310 (as described above and in the present disclosure).

The health engine 310 may also include a health recommendation scoring engine (e.g., health recommendation scoring engine 316). Alternatively or in addition, the health engine 310 may be configurable or configured to perform the functions, processes, and/or actions of a health recommendation scoring engine 316. The health recommendation scoring engine 316 may be configurable or configured to establish, derive, and/or calculate a score (e.g., a first score) for each of the health-related recommendations selected for each user by the health engine 310. The establishing, deriving, and/or calculating of the score for each health-related recommendation selected for each user may be based on a predetermined criterion. For example, the establishing, deriving, and/or calculating of the score for each health-related recommendation selected for each user may be based on, among other things, availability, geographical location, etc. of the recommendation. As another example, the establishing, deriving, and/or calculating of the score for each health-related recommendation selected for each user may be based on, among other things, the health-related information selected for the user by the health engine 310 (as described above and in the present disclosure).

The health engine 310 may also include a health recommendation ranking engine (e.g., health recommendation ranking engine 318). Alternatively or in addition, the health engine 310 may be configurable or configured to perform the functions, processes, and/or actions of a health recommendation ranking engine 318. The health recommendation ranking engine 318 may be configurable or configured to ranking the health-related recommendations selected for each user by the health engine 310. The ranking of the health-related recommendations selected for each user may result or arrive at the first ranked list for each user. The ranking of the health-related recommendations selected for each user may be based on a predetermined criterion. For example, the ranking of the health-related recommendations selected for each user may be based on, among other things, the health-related information selected for each user by the health engine 310 (as described above and in the present disclosure). As another example, the ranking of the health-related recommendations selected for each user may be based on, among other things, the score established, derived, and/or calculated for each health-related recommendation selected for each user.

The health engine 310 may then be configurable or configured to derive the first ranked list for each user based on the ranking of the health-related recommendations selected for each user by the health engine 310. It is to be understood in the present disclosure that the functions, processes, and/or actions performed by and/or for the health engine 310, as described above and in the present disclosure, including the selecting of health-related information for each user, searching the recommendations database 360, selecting health-related recommendations for each user, establishing, deriving, and/or calculating a score for each of the health-related recommendations selected for each user, and/or ranking of the health-related recommendations selected for each user, may be performed in the order described above or in a different order without departing from the teachings of the present disclosure.

The Financial Engine (e.g., Financial Engine 320).

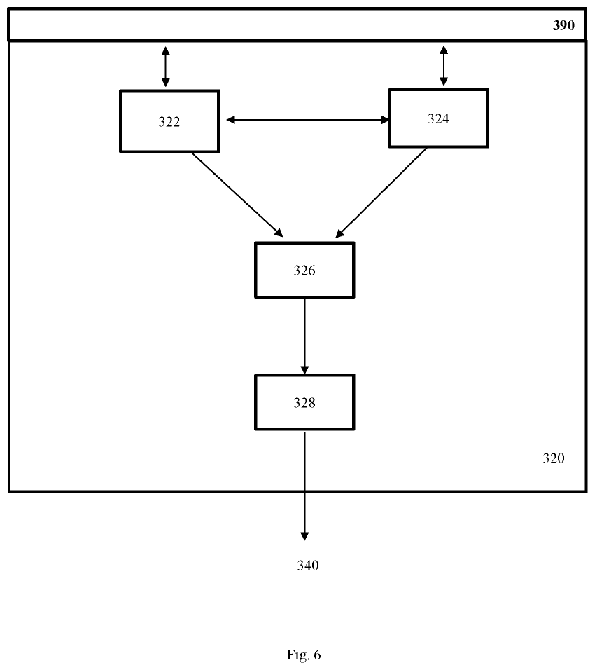

As illustrated in at least FIG. 4 and FIG. 6, the main processor 300a may include a financial engine (e.g., financial engine 320). Alternatively or in addition, the main processor 300a may be configurable or configured to perform the functions, processes, and/or actions of a financial engine 320.

The financial engine 320 may be configurable or configured to derive a second ranked list for one or more users. As will be further described in the present disclosure, the second ranked list derived for each user may then be used by, among other elements of the main processor 300a, the final recommendation engine 340 to arrive at a final ranked list of recommendations for each user.

The financial engine 320 may include a financial information selection engine (e.g., financial information selection engine 322). Alternatively or in addition, the financial engine 320 may be configurable or configured to perform the functions, processes, and/or actions of a financial information selection engine 322. The financial information selection engine 322 may be configurable or configured to select financial-related information for each user. Such information selected may be from among the information received for (or pertaining to or about) the user (e.g., information obtained/received/requested by the communication processor 380, as described above and in the present disclosure).

The financial engine 320 may also include a financial recommendation selection engine (e.g., financial recommendation selection engine 324). Alternatively or in addition, the financial engine 320 may be configurable or configured to perform the functions, processes, and/or actions of a financial recommendation selection engine 324. The financial recommendation selection engine 324 may be configurable or configured to search the recommendations database 360 (as described above and in the present disclosure).

The financial recommendation selection engine 324 may also be configurable or configured to select, based on the searching of the recommendations database 360, financial-related recommendations for each user. The financial-related recommendations for each user may be selected based on a predetermined criterion. For example, the financial-related recommendation for each user may be selected based on, among other things, availability, geographical location, etc. of the recommendation. As another example, the financial-related recommendation for each user may be selected from a financial perspective. As another example, the financial-related recommendation for each user may be selected based on, among other things, the financial-related information selected for each user by the financial engine 320 (as described above and in the present disclosure).

The financial engine 320 may also include a financial recommendation scoring engine (e.g., financial recommendation scoring engine 326). Alternatively or in addition, the financial engine 320 may be configurable or configured to perform the functions, processes, and/or actions of a financial recommendation scoring engine 326. The financial recommendation scoring engine 326 may be configurable or configured to establish, derive, and/or calculate a score (e.g., a second score) for each of the financial-related recommendations selected for each user by the financial engine 320. The establishing, deriving, and/or calculating of the score for each financial-related recommendation selected for each user may be based on a predetermined criterion. For example, the establishing, deriving, and/or calculating of the score for each financial-related recommendation selected for each user may be based on, among other things, availability, geographical location, etc. of the recommendation. As another example, the establishing, deriving, and/or calculating of the score for each financial-related recommendation selected for each user may be based on, among other things, the financial-related information selected for the user by the financial engine 320 (as described above and in the present disclosure).

The financial engine 320 may also include a financial recommendation ranking engine (e.g., financial recommendation ranking engine 328). Alternatively or in addition, the financial engine 320 may be configurable or configured to perform the functions, processes, and/or actions of a financial recommendation ranking engine 328. The financial recommendation ranking engine 328 may be configurable or configured to rank the financial-related recommendations selected for each user by the financial engine 320. The ranking of the financial-related recommendations selected for each user may result or arrive at the second ranked list for each user. The ranking of the financial-related recommendations selected for each user may be based on a predetermined criterion. For example, the ranking of the financial-related recommendations selected for each user may be based on, among other things, the financial-related information selected for each user by the financial engine 320 (as described above and in the present disclosure). As another example, the ranking of the financial-related recommendations selected for each user may be based on, among other things, the score established, derived, and/or calculated for each financial-related recommendation selected for each user.

The financial engine 320 may then be configurable or configured to derive the second ranked list for each user based on the ranking of the financial-related recommendations selected for each user by the financial engine 320. It is to be understood in the present disclosure that the functions, processes, and/or actions performed by and/or for the financial engine 320, as described above and in the present disclosure, including the selecting of financial-related information for each user, searching the recommendations database 360, selecting financial-related recommendations for each user, establishing, deriving, and/or calculating a score for each of the financial-related recommendations selected for each user, and/or ranking of the financial-related recommendations selected for each user, may be performed in the order described above or in a different order without departing from the teachings of the present disclosure.

The Prediction Engine (e.g., Prediction Engine 330).

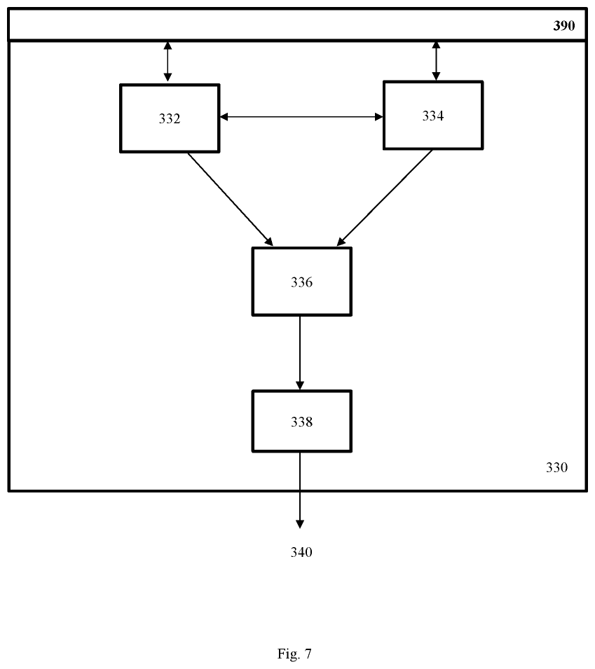

As illustrated in at least FIG. 4 and FIG. 7, the main processor 300a may include a prediction engine (e.g., prediction engine 330). Alternatively or in addition, the main processor 300a may be configurable or configured to perform the functions, processes, and/or actions of a prediction engine 330.

The prediction engine 330 may be configurable or configured to derive a third ranked list for one or more users. As will be further described in the present disclosure, the third ranked list derived for each user may then be used by, among other elements of the main processor 300a, the final recommendation engine 340 to arrive at a final ranked list of recommendations for each user.

The prediction engine 330 may include a prediction-based information selection engine (e.g., prediction-based information selection engine 332). Alternatively or in addition, the prediction engine 330 may be configurable or configured to perform the functions, processes, and/or actions of a prediction-based information selection engine 332. The prediction-based information selection engine 332 may be configurable or configured to select one or more historical or prediction-based information for each user, such as from a historical selections database 362. For example, the historical information selected may include historically accepted information, including information pertaining to recommendations previously provided to each user that were previously accepted or selected by each user. As another example, the historical information selected may include historically not accepted information, including information pertaining to recommendations previously provided to each user that were not previously accepted or selected by each user. As another example, the historic information selected may include historically not recommended information, including information pertaining to recommendations that were not previously provided to each user.

The prediction engine 330 may also include a prediction-based recommendation selection engine (e.g., prediction-based recommendation selection engine 334). Alternatively or in addition, the prediction engine 330 may be configurable or configured to perform the functions, processes, and/or actions of a prediction-based recommendation selection engine 334. The prediction-based recommendation selection engine 334 may be configurable or configured to search the recommendations database 360 (as described above and in the present disclosure).

The prediction-based recommendation selection engine 334 may also be configurable or configured to select, based on the searching of the recommendations database 360, prediction-related or prediction-based recommendations for each user. The prediction-related or prediction-based recommendations for each user may be selected based on a predetermined criterion. For example, the prediction-related or prediction-based recommendation for each user may be selected based on, among other things, availability, geographical location, etc. of the recommendation. As another example, the prediction-related or prediction-based recommendation for each user may be selected from a historical, prediction-based, or prediction-related perspective. As another example, the prediction-related or prediction-based recommendation for each user may be selected based on, among other things, the historical and/or prediction-based information selected for each user by the prediction engine 330 (as described above and in the present disclosure). As another example, the prediction-related or prediction-based recommendations for each user may be selected based a likelihood of being selected by the first user.

The prediction engine 330 may also include a prediction-based recommendation scoring engine (e.g., prediction-based recommendation scoring engine 336). Alternatively or in addition, the prediction engine 330 may be configurable or configured to perform the functions, processes, and/or actions of a prediction-based recommendation scoring engine 336. The prediction-based recommendation scoring engine 336 may be configurable or configured to establish, derive, and/or calculate a score (e.g., a third score) for each of the prediction-related or prediction-based recommendations selected for each user by the prediction engine 330. The establishing, deriving, and/or calculating of the score for each prediction-related or prediction-based recommendation selected for each user may be based on a predetermined criterion. For example, the establishing, deriving, and/or calculating of the score for each prediction-related or prediction-based recommendation selected for each user may be based on, among other things, availability, geographical location, etc. of the recommendation. As another example, the establishing, deriving, and/or calculating of the score for each prediction-related or prediction-based recommendation selected for each user may be based on, among other things, the historical or prediction-based information selected for the user by the prediction engine 330 (as described above and in the present disclosure).

The prediction engine 330 may also include a prediction-based recommendation ranking engine (e.g., prediction-based recommendation ranking engine 338). Alternatively or in addition, the prediction engine 330 may be configurable or configured to perform the functions, processes, and/or actions of a prediction-based recommendation ranking engine 338. The prediction-based recommendation ranking engine 338 may be configurable or configured to rank the prediction-related or prediction-based recommendations selected for each user by the prediction engine 330. The ranking of the prediction-related or prediction-based recommendations selected for each user may result or arrive at the third ranked list for each user. The ranking of the prediction-related or prediction-based recommendations selected for each user may be based on a predetermined criterion. For example, the ranking of the prediction-related or prediction-based recommendations selected for each user may be based on, among other things, the historical or prediction-based information selected for each user by the prediction engine 330 (as described above and in the present disclosure). As another example, the ranking of the prediction-related or prediction-based recommendations selected for each user may be based on, among other things, the score established, derived, and/or calculated for each prediction-related or prediction-based recommendation selected for each user.

The prediction engine 330 may then be configurable or configured to derive the third ranked list for each user based on the ranking of the prediction-related or prediction-based recommendations selected for each user by the prediction engine 330. It is to be understood in the present disclosure that the functions, processes, and/or actions performed by and/or for the prediction engine 330, as described above and in the present disclosure, including the selecting of historical or prediction-based information for each user, searching the recommendations database 360, selecting prediction-related or prediction-based recommendations for each user, establishing, deriving, and/or calculating a score for each of the prediction-related or prediction-based recommendations selected for each user, and/or ranking of the prediction-related or prediction-based recommendations selected for each user, may be performed in the order described above or in a different order without departing from the teachings of the present disclosure.

The Final recommendation Engine (e.g., Final Recommendation Engine 340).

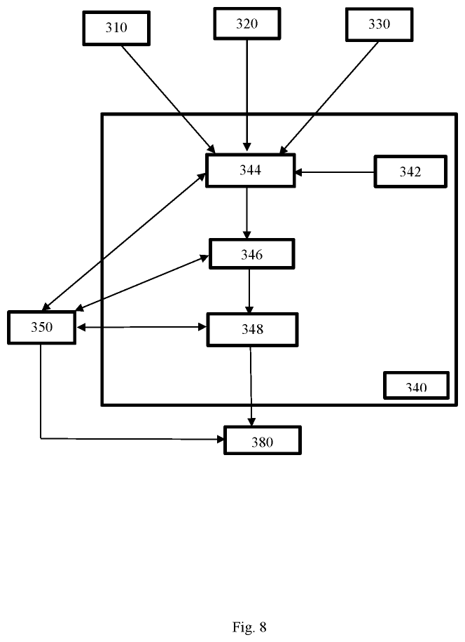

As illustrated in at least FIG. 4 and FIG. 8, the main processor 300a may include a final recommendation engine (e.g., final recommendation engine 340). Alternatively or in addition, the main processor 300a may be configurable or configured to perform the functions, processes, and/or actions of a final recommendation engine 340.

The final recommendation engine 340 may be configurable or configured to derive a final ranked list for one or more users. The final ranked list derived for each user may be used by, among other elements of the main processor 300a, the final recommendation engine 340 to arrive at and display to each user a final ranked list of recommendations for each user.

The final recommendation engine 340 may include a predetermined criterion selector (e.g., predetermined criterion selector 342). Alternatively or in addition, the final recommendation engine 340 may be configurable or configured to perform the functions, processes, and/or actions of a predetermined criterion selector 342. The predetermined criterion selector 342 may be configurable or configured to select one or more predetermined criterion for use in deriving the final ranked list for each user. Such predetermined criterion may include, but not limited to, scaling, weighting, normalizing, adjusting, and/or offsetting operations. For example, the predetermined criterion may apply an equal weight for the first ranked list (health-based), second ranked list (financial-based), and third ranked list (prediction-based). As another example, the predetermined criterion may apply a zero weight (or elimination) of the third ranked list (prediction-based). As another example, the predetermined criterion may apply a 40% weight to the first ranked list (health-based), 30% weight to the second ranked list (financial-based), and a 30% weight to the third ranked list (prediction-based).

The final recommendation engine 340 may also include a final recommendation selection engine (e.g., final recommendation selection engine 344). Alternatively or in addition, the final recommendation engine 340 may be configurable or configured to perform the functions, processes, and/or actions of a final recommendation selection engine 344. The final recommendation selection engine 344 may be configurable or configured to retrieve the first ranked list, the second ranked list, and/or the third ranked list (as described above and in the present disclosure) based on, among other things, the predetermined criterion selected by the predetermined criterion selector 342 and/or a health score for each user provided by the health score engine 350. For example, the final recommendation engine 340 may be configurable or configured to retrieve the first ranked list, the second ranked list, and the third ranked list. As another example, the final recommendation engine 340 may be configurable or configured to retrieve the first ranked list and the second ranked list. As another example, the final recommendation engine 340 may be configurable or configured to retrieve the first ranked list and the third ranked list. As another example, the final recommendation engine 340 may be configurable or configured to retrieve the second ranked list and the third ranked list.

The final recommendation selection engine 344 may also be configurable or configured to process the retrieved first ranked list, second ranked list, and/or third ranked list based on, among other things, the predetermined criterion selected by the predetermined criterion selector 342 and/or a health score for each user provided by the health score engine 350. The final recommendation selection engine 344 may also be configurable or configured to select, based on the processing of the final recommendation selection engine 344, final recommendations from the retrieved first ranked list, second ranked list, and/or third ranked list.

The final recommendation engine 340 may also include a final recommendation scoring engine (e.g., final recommendation scoring engine 346). Alternatively or in addition, the final recommendation engine 340 may be configurable or configured to perform the functions, processes, and/or actions of a final recommendation scoring engine 346. The final recommendation scoring engine 346 may be configurable or configured to establish, derive, and/or calculate a score (e.g., a final score) for each of the final recommendations selected for each user by the final recommendation selection engine 344 based on, among other things, the predetermined criterion selected by the predetermined criterion selector 342 and/or a health score for each user provided by the health score engine 350.

The final recommendation engine 340 may also include a final recommendation ranking engine (e.g., final recommendation ranking engine 348). Alternatively or in addition, the final recommendation engine 340 may be configurable or configured to perform the functions, processes, and/or actions of a final recommendation ranking engine 348. The final recommendation ranking engine 348 may be configurable or configured to rank the final recommendations selected for each user by the final recommendation selection engine 344 based on, among other things, the predetermined criterion selected by the predetermined criterion selector 342 and/or a health score for each user provided by the health score engine 350. The ranking of the final recommendations selected for each user may result or arrive at the final ranked list for each user. The ranking of the final recommendations selected for each user may be based on a predetermined criterion. For example, the ranking of the final recommendations selected for each user may be based on, among other things, the score established, derived, and/or calculated for each final recommendation selected for each user.

The final recommendation engine 340 may then be configurable or configured to derive the final ranked list for each user based on, among other things, the ranking of the final recommendations selected for each user by the final recommendation selection engine 344. The final recommendation engine 340 may then be configurable or configured to communicate with the communication processor 380 to display, on a graphical display of each user, the final ranked list of recommendations for each user. It is to be understood in the present disclosure that the functions, processes, and/or actions performed by and/or for the final recommendation engine 340, as described above and in the present disclosure, including the selecting of predetermined criterion, retrieving of the first ranked list, second ranked list, and/or third ranked list, processing of the first ranked list, second ranked list, and/or third ranked list based on the predetermined criterion, selecting final recommendations, establishing, deriving, and/or calculating a final score for each of the final recommendations selected for each user, and/or ranking of the final recommendations selected for each user, may be performed in the order described above or in a different order without departing from the teachings of the present disclosure.

Health Score Engine (e.g., Health Score Engine 350).

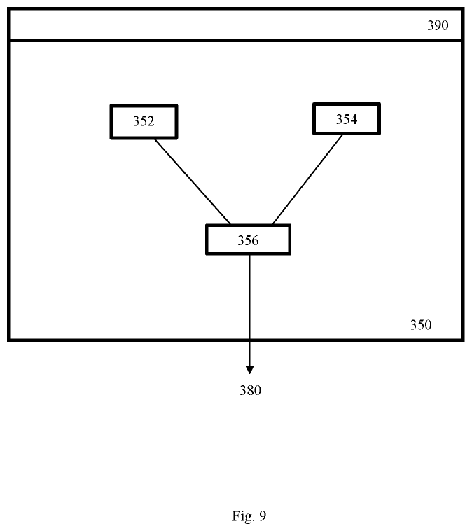

As illustrated in at least FIG. 4 and FIG. 9, the main processor 300a may include a health score engine (e.g., health score engine 350). Alternatively or in addition, the main processor 300a may be configurable or configured to perform the functions, processes, and/or actions of a health score engine 350.

The health score engine 350 may be configurable or configured to establish, derive, and/or calculate a health score for each user. The health score derived for each user may be a score indicative of, estimating, and/or measuring an overall health of each user. The health score derived for each user may also be used by, among other elements of the main processor 300a, the final recommendation engine 340 to arrive at a final ranked list of recommendations for each user. The health score engine 350 may also be configurable or configured to establish, derive, and/or calculate, for each user, an achievable health score for each recommendation in the first ranked list, the second ranked list, the third ranked list, and/or the final ranked list of recommendations.

The health score engine 350 may include a metabolic scoring engine 352. The metabolic scoring engine 352 may be configurable or configured to establish, derive, and/or calculate a metabolic score for each user. The metabolic scoring engine 352 may be configurable or configured to transform metabolic information for each user into a metabolic score. In example embodiments, metabolic information may include, but is not limited to, BMI, glucose, triglyceride, HDL cholesterol, and blood pressure information for each user.

The health score engine 350 may include a lifestyle scoring engine 354. The lifestyle scoring engine 354 may be configurable or configured to establish, derive, and/or calculate a lifestyle score for each user. The lifestyle scoring engine 354 may be configurable or configured to transform lifestyle information for each user into a lifestyle score. In example embodiments, lifestyle information may include, but is not limited to, health risk assessment (HRA) for each user, number of steps by each user (e.g., daily count of steps), and images of each user.

The health score derived for each user may be established, derived, and/or calculated based on the metabolic score, as established, derived, and/or calculated by the metabolic scoring engine 352, and the lifestyle score, as established, derived, and/or calculated by the lifestyle scoring engine 354. Specifically, the health score engine 350 may include a health scorer 356 configured to establish, derive, and/or calculate the health score based on the metabolic score and the lifestyle score.

User Information Database (e.g., User Information Database 366).

The system 300 may also include one or more user information databases (e.g., user information database 366). The user information database 366 may be configurable or configured to store a plurality of information obtained for, by, from, and/or pertaining to the users. The user information database 366 may include personal information, personal identification, health-related information, activity-related information, physical-related information, sick-leave-related information, health-related insurance information, general wellbeing related information, fitness-related information, user preference related information, employee financial information, employee communication, geolocation information; life-based life work event (LWE) information of the employee such as martial status, family nucleus, employee HR details such as date of joining the company, current designation in the company, pay level and entitlements and any other related information which may be used for analysis. Examples of information that may be stored in the user information database 366 include information related to physical attributes (e.g., age, sex, height, weight, body mass index (BMI), etc.), health screening results (e.g., blood sugar levels, triglyceride levels, blood pressure information etc.), medical records, conditions or illnesses (e.g., cardiovascular diseases, diabetes, cancer, etc.), work-based life work event (LWE) information (e.g., marital status, family nucleus, moving houses, etc.) work related information (e.g., income bracket, salary, dependents, duration of sick leaves, medical certificates, etc.), activity related information (e.g., number of steps taken, number of hours being idle, number of hours of movement, etc.) and insurance related information (e.g., types of benefits, insurance claims and amount, type of illnesses covered, exclusions, etc.). It is to be understood in the present disclosure that the user information database 366 may include information obtained/received/requested by the communication processor 380 (as described above and in the present disclosure), including life work events (LWE) for one or more users such as personal life-related changes (e.g., marriage, divorce, having a baby, adopting a child, a death in the family, illness or injury to the user, family member, friend, etc.) and work or employment-related changes (e.g., new employment, a change in employment, loss of employment, changes to income/remuneration (e.g., changes that can result in increment or decrement that affects insurance coverage that the user qualifies for), changes to employment category (e.g., changes that can affect coverage the user qualifies for), etc.).

Example Embodiments of a Method for Processing and/or Managing Health-Related Information (e.g., Method 1000)

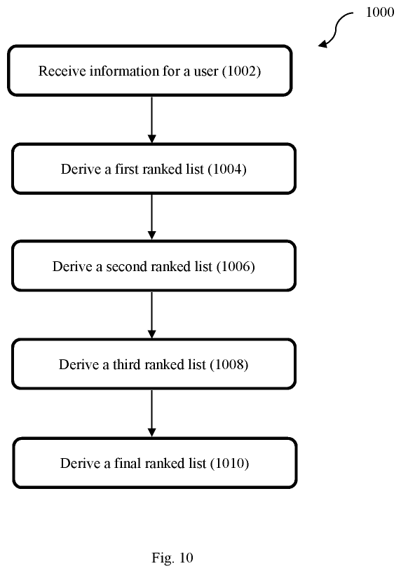

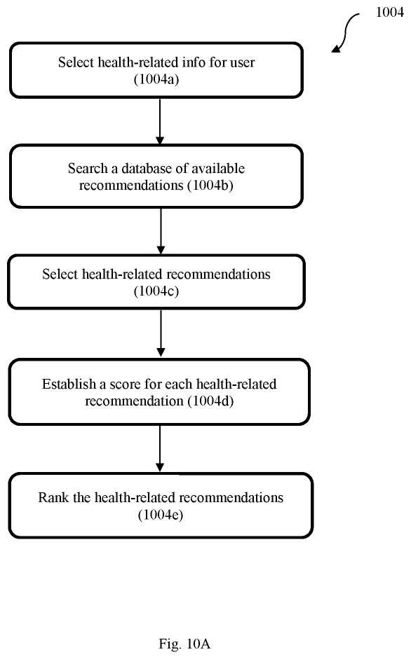

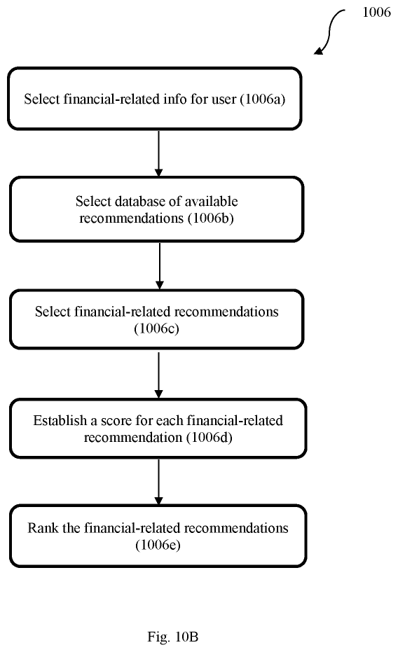

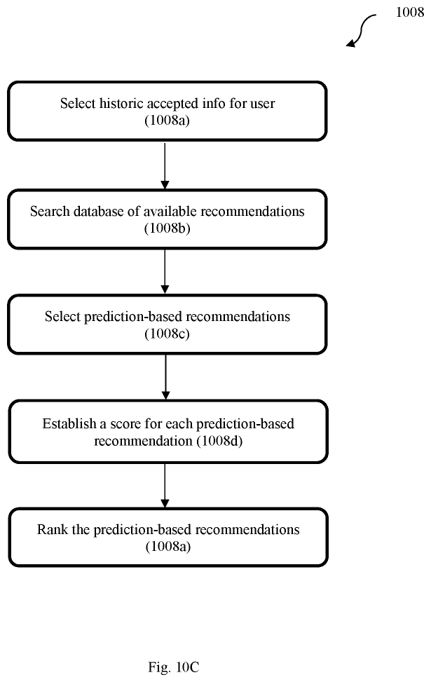

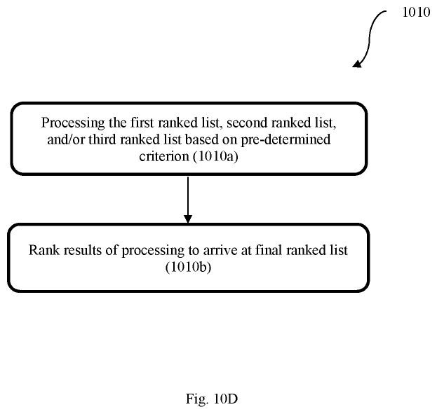

FIG. 10 illustrates an example embodiment of a method (e.g., method 1000) for processing and/or managing health-related information. The method 1000 may be performable by example embodiments of the system 300, main processor 300a, and/or elements thereof, as described above and in the present disclosure. The method 1000 may be configurable and/or configured to derive one or more ranked lists of recommendations for one or more users. The method 1000 may include establishing one or more communication channels 390. The method 1000 may also include receiving information for a user (e.g., action 1002). The method 1000 may also include deriving a first ranked list of recommendations (e.g., action 1004). The method 1000 may also include deriving a second ranked list of recommendations (e.g., action 1006). The method 1000 may also include deriving a third ranked list of recommendations (e.g., action 1008). The method 1000 may also include deriving a final ranked list of recommendations (e.g., action 1010).

Example embodiments of the method 1000 of processing and/or managing health-related information may include or not include one or more of the actions described above and in the present disclosure, may include additional actions, operations, and/or functionality, may be performed in different sequences and/or combinations, and/or one or more of the actions, operations, and/or functionality may be combinable into a single action, operation, and/or functionality and/or divided into two or more actions, operations, and/or functionalities. The method 1000 of processing and/or managing health-related information, and actions and elements thereof, will now be further explained with reference to the accompanying figures.

Establishing a Communication Channel.

In an example embodiment, the method 1000 may include establishing one or more communication channels. Each communication channel may be established by the main processor 300a. For example, a communication channel 390 may be established between the main processor 300a and a user information database 366. As another example, a communication channel 390 may be established between the main processor 300a and an employee 370. As another example, a communication channel 390 may be established between the main processor 300a and an employer 371. As another example, a communication channel 390 may be established between the main processor 300a and an insurance company 372. As another example, a communication channel 390 may be established between the main processor 300a and a medical provider 373. As another example, a communication channel 390 may be established between the main processor 300a and a wellness provider 374. As another example, a communication channel 390 may be established between the main processor 300a and a retailer 375. As another example, a communication channel 390 may be established between the main processor 300a and other providers 376. As another example, a communication channel 390 may be established between the main processor 300a and a historical selections database 362. As another example, a communication channel 390 may be established between the main processor 300a and other databases 364.

Receiving Information for a User (e.g., Action 1002).

In an example embodiment, the method 1000 may include receiving information for one or more users (e.g., action 1002). The information for each user may be received by the main processor 300a via one or more of the communication channels 390. The information for each user received by the main processor 300a may be information for, from, and/or pertaining to the user. It is to be understood in the present disclosure that the information received by the main processor 300a may include life work events (LWE) for one or more users, such as personal life-related changes (e.g., marriage, divorce, having a baby, adopting a child, a death in the family, illness or injury to the user, family member, friend, etc. and work or employment-related changes (e.g., new employment, a change in employment, loss of employment, changes to income/remuneration (e.g., changes that can result in increment or decrement that affects insurance coverage that the user qualifies for), changes to employment category (e.g., changes that can affect coverage the user qualifies for), etc.).

Deriving a First Ranked List (e.g., Action 1004).

As illustrated in at least FIG. 10 and FIG. 10A, an example embodiment of the method 1000 may include deriving a first ranked list for one or more users (e.g., action 1004). The first ranked list may be a list of recommendations (e.g., from a health perspective) for each user. The deriving of the first ranked list of recommendations 1004 may be performable by example embodiments of the health engine 310, as described in the present disclosure.

The deriving of the first ranked list 1004 may include selecting health-related information for each user (e.g., action 1004a). The health-related information selected for each user may be from among information received for the user (e.g., information received in action 1002). The selecting of the health-related information 1004a may be performable by example embodiments of the health information selection engine 312, as described above and in the present disclosure. The deriving of the first ranked list 1004 may also include searching a database of recommendations (e.g., recommendations database 360, as described in the present disclosure) (e.g., action 1004b). The deriving of the first ranked list 1004 may also include selecting health-related recommendations (e.g., 1004c). The health-related recommendations may be selected based on the searching of the recommendations database (e.g., searching in action 1004b). The selected health-related recommendations may be or include recommendations selected from a health perspective. The searching of the recommendations database 360 and/or selecting of the health-related recommendations 1004b, 1004c may be performable by example embodiments of the health recommendation selection engine 314, as described above and in the present disclosure. The deriving of the first ranked list 1004 may also include establishing, for each user, a score (e.g., a first score) for each of the selected health-related recommendations (e.g., action 1004d). Such establishing of the score may be based on or more of the selected health-related information for each user (e.g., selected in action 1004a). The establishing of the score for each selected health-related recommendation may be performable by example embodiments of the health recommendation scoring engine 316, as described above and in the present disclosure. The deriving of the first ranked list 1004 may also include ranking the selected health-related recommendations (e.g., action 1004e). The ranking of the selected health-related recommendations may result in the first ranked list of recommendations for each user. The ranking of the selected health-related recommendations to arrive at the first ranked list for each user may be based on the score of each of the selected health-related recommendations for the user. The ranking of the selected health-related recommendations to arrive at the first ranked list of recommendations may be performable by example embodiments of the health recommendation ranking engine 318, as described above and in the present disclosure.

Deriving a Second Ranked List (e.g., Action 1006).

As illustrated in at least FIG. 10 and FIG. 10B, an example embodiment of the method 1000 may include deriving a second ranked list for one or more users (e.g., action 1006). The second ranked list may be a list of recommendations (e.g., from a financial perspective) for each user. The deriving of the second ranked list of recommendations 1006 may be performable by example embodiments of the financial engine 320, as described in the present disclosure.

The deriving of the second ranked list 1006 may include selecting financial-related information for each user (e.g., action 1006a). The financial-related information selected for each user may be from among information received for the user (e.g., information received in action 1002). The selecting of the financial-related information 1006a may be performable by example embodiments of the financial information selection engine 322, as described above and in the present disclosure. The deriving of the second ranked list 1006 may also include searching a database of recommendations (e.g., recommendations database 360, as described in the present disclosure) (e.g., action 1006b). The deriving of the second ranked list 1006 may also include selecting financial-related recommendations (e.g., 1006c). The financial-related recommendations may be selected based on the searching of the recommendations database (e.g., searching in action 1006b). The selected financial-related recommendations may be or include recommendations selected from a financial perspective. The searching of the recommendations database 160 and/or selecting of the financial-related recommendations 1006b, 1006c may be performable by example embodiments of the financial recommendation selection engine 324, as described above and in the present disclosure. The deriving of the second ranked list 1006 may also include establishing, for each user, a score (e.g., a second score) for each of the selected financial-related recommendations (e.g., action 1006d). Such establishing of the score may be based on or more of the selected financial-related information for each user (e.g., selected in action 1006a). The establishing of the score for each selected financial-related recommendation may be performable by example embodiments of the financial recommendation scoring engine 326, as described above and in the present disclosure. The deriving of the second ranked list 1006 may also include ranking the selected financial-related recommendations (e.g., action 1006e). The ranking of the selected financial-related recommendations may result in the second ranked list of recommendations for each user. The ranking of the selected financial-related recommendations to arrive at the second ranked list for each user may be based on the score of each of the selected financial-related recommendations for the user. The ranking of the selected financial-related recommendations to arrive at the second ranked list of recommendations may be performable by example embodiments of the financial recommendation ranking engine 328, as described above and in the present disclosure.

Deriving a Third Ranked List (e.g., Action 1008).

As illustrated in at least FIG. 10 and FIG. 10C, an example embodiment of the method 1000 may include deriving a third ranked list for one or more users (e.g., action 1008). The third ranked list may be a list of recommendations (e.g., from a historical or prediction-based perspective) for each user. The deriving of the third ranked list of recommendations 1008 may be performable by example embodiments of the prediction engine 330, as described in the present disclosure.

The deriving of the third ranked list 1008 may include selecting historical or prediction-based information for each user (e.g., action 1008a). The historical or prediction-based information selected for each user may be from among information received for the user (e.g., information received in action 1002). For example, the historical information selected may include historically accepted information, including information pertaining to recommendations previously provided to each user that were previously accepted or selected by each user. As another example, the historical information selected may include historically not accepted information, including information pertaining to recommendations previously provided to each user that were not previously accepted or selected by each user. As another example, the historic information selected may include historically not recommended information, including information pertaining to recommendations that were not previously provided to each user. The selecting of the historical or prediction-based information 1008a may be performable by example embodiments of the prediction-based information selection engine 332, as described above and in the present disclosure. The deriving of the third ranked list 1008 may also include searching a database of recommendations (e.g., recommendations database 360, as described in the present disclosure) (e.g., action 1008b). The deriving of the third ranked list 1008 may also include selecting prediction-based or prediction-related recommendations (e.g., 1008c). The prediction-based or prediction-related recommendations may be selected based on the searching of the recommendations database (e.g., searching in action 1008b). The selected prediction-based or prediction-related recommendations may be or include recommendations selected from a historical or prediction-based perspective. The searching of the recommendations database 360 and/or selecting of the prediction-based or prediction-related recommendations 1008b, 1008c may be performable by example embodiments of the prediction-based recommendation selection engine 334, as described above and in the present disclosure. The deriving of the third ranked list 1008 may also include establishing, for each user, a score (e.g., a third score) for each of the selected prediction-based or prediction-related recommendations (e.g., action 1008d). Such establishing of the score may be based on or more of the selected historical or prediction-based information for each user (e.g., selected in action 1008a). The establishing of the score for each selected prediction-based or prediction-related recommendation may be performable by example embodiments of the prediction-based recommendation scoring engine 336, as described above and in the present disclosure. The deriving of the third ranked list 1008 may also include ranking the selected prediction-based or prediction-related recommendations (e.g., action 1008e). The ranking of the selected prediction-based or prediction-related recommendations may result in the third ranked list of recommendations for each user. The ranking of the selected prediction-based or prediction-related recommendations to arrive at the third ranked list for each user may be based on the score of each of the selected prediction-based or prediction-related recommendations for the user. The ranking of the selected prediction-based or prediction-related recommendations to arrive at the third ranked list of recommendations may be performable by example embodiments of the prediction-based recommendation ranking engine 338, as described above and in the present disclosure.

Deriving a Final Ranked List (e.g., Action 1010).

As illustrated in at least FIG. 10 and FIG. 10D, an example embodiment of the method 1000 may include deriving a final ranked list for one or more users (e.g., action 1010). The final ranked list derived for each user may be used by, among other elements of the main processor 300a, the final recommendation engine 340 to arrive at and display to each user a final ranked list of recommendations for each user. The deriving of the final ranked list of recommendations 1010 may be performable by example embodiments of the final recommendation engine 340, as described in the present disclosure.

The deriving of the final ranked list 1010 may include processing the first ranked list, the second ranked list, and/or the third ranked list based on one or more predetermined criterion (e.g., action 1010a). Such predetermined criterion may include, but not limited to, scaling, weighting, normalizing, adjusting, and/or offsetting operations. For example, the predetermined criterion may apply an equal weight for the first ranked list (health-based), second ranked list (financial-based), and third ranked list (prediction-based). As another example, the predetermined criterion may apply a zero weight (or elimination) of the third ranked list (prediction-based). As another example, the predetermined criterion may apply a 40% weight to the first ranked list (health-based), 30% weight to the second ranked list (financial-based), and a 30% weight to the third ranked list (prediction-based). As another example, the predetermined criterion may include the health score, as described above and in the present disclosure. The predetermined criterion may be selected by example embodiments of the predetermined criterion selector 342, as described above and in the present disclosure. The processing of the first ranked list, second ranked list, and third ranked list based on one or more predetermined criterion 1010a may be performable by example embodiments of the final recommendation selection engine 344. The deriving of the final ranked list 1010 may include selecting, based on the processing 1010a, final recommendations from the first ranked list, second ranked list, and/or third ranked list.

The deriving of the final ranked list 1010 may also include establishing, for each user, a score (e.g., a final score) for each of the selected final recommendations. Such establishing of the final score may be based on or more of the predetermined criterion and/or the health score. The establishing of the final score for each selected final recommendation may be performable by example embodiments of the final recommendation scoring engine 346, as described above and in the present disclosure. The deriving of the final ranked list 1010 may also include ranking the selected final recommendations (e.g., action 1010b). The ranking of the selected final recommendations may result in the final ranked list of recommendations for each user. The ranking of the selected final recommendations to arrive at the final ranked list for each user may be based on the final score of each of the selected final recommendations for the user. The ranking of the selected final recommendations to arrive at the final ranked list of recommendations may be performable by example embodiments of the final recommendation ranking engine 348, as described above and in the present disclosure.

Example Embodiments of a Method for Processing and/or Managing Health-Related Information (e.g., Method 1100)

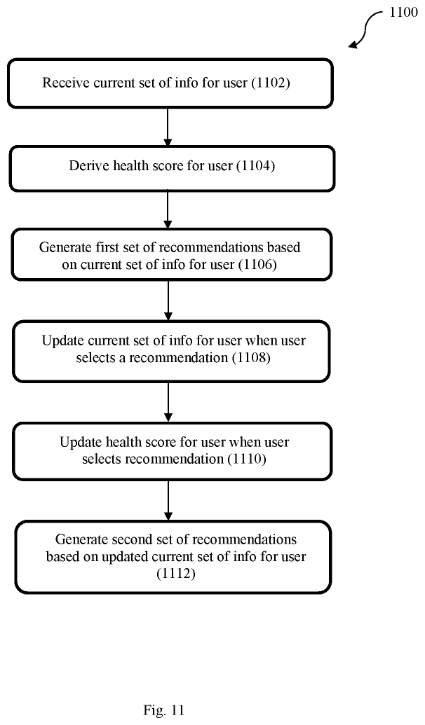

FIG. 11 illustrates an example embodiment of a method (e.g., method 1100) for processing and/or managing health-related information. The method 1100 may be performable by example embodiments of the system 300, main processor 300a, and/or elements thereof, as described above and in the present disclosure. The method 1100 may be configurable and/or configured to establish, derive, and/or calculate a health score for each user. The health score derived for each user may be a score indicative of, estimating, and/or measuring an overall health of each user. The health score derived for each user may also be used to arrive at a final ranked list of recommendations for each user. The method 1100 may be for use to establish, derive, and/or calculate, for each user, an achievable health score for each recommendation in the first ranked list, the second ranked list, the third ranked list, and/or the final ranked list of recommendations.

The method 1100 may include establishing one or more communication channels 390, as described above and in the present disclosure. The method 1100 may also include receiving a current set of information for a user (e.g., via the one or more communication channels 390, as described above and in the present disclosure) (e.g., action 1102). The method 1100 may also include deriving a health score for the user (e.g., action 1104). The deriving of the health score for the user may be performable by example embodiments of the health score engine 350. The method 1100 may also include generating a first set of recommendations for the user based on the current set of information for the user (e.g., the final ranked list of recommendations, as described above and in the present disclosure) (e.g., action 1106). The method 1100 may also include updating the current set of information for the user when the user selects a recommendation (e.g., when the user selects from the final ranked list of recommendations) (e.g., action 1108). The method 1100 may also include updating the health score for the user (e.g., the health score as derived in action 1104) when the user selects a recommendation (e.g., from the first set of recommendations) (e.g., action 1110). The method 1100 may also include generating a second set of recommendations based on the updated current set of information for the user (as updated in action 1108).

Example embodiments of the method 1100 of processing and/or managing health-related information may include or not include one or more of the actions described above and in the present disclosure, may include additional actions, operations, and/or functionality, may be performed in different sequences and/or combinations, and/or one or more of the actions, operations, and/or functionality may be combinable into a single action, operation, and/or functionality and/or divided into two or more actions, operations, and/or functionalities.

Example Embodiments of a Method for Processing and/or Managing Health-Related Information (e.g., Method 1200)

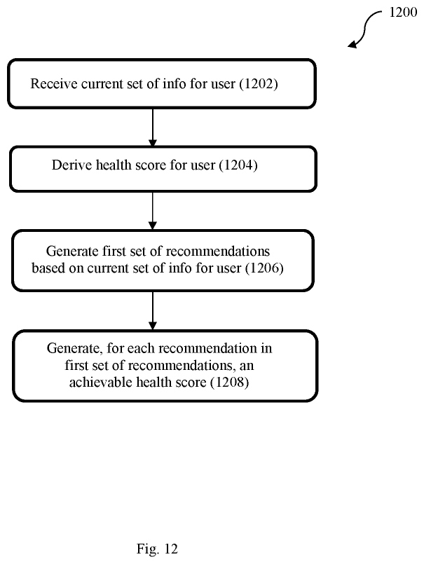

FIG. 12 illustrates an example embodiment of a method (e.g., method 1200) for processing and/or managing health-related information. The method 1200 may be performable by example embodiments of the system 300, main processor 300a, and/or elements thereof, as described above and in the present disclosure. The method 1200 may be configurable and/or configured to establish, derive, and/or calculate a health score for each user, as described above and in the present disclosure.

The method 1200 may include establishing one or more communication channels 390, as described above and in the present disclosure. The method 1200 may also include receiving a current set of information for a user (e.g., via the one or more communication channels 390, as described above and in the present disclosure) (e.g., action 1202). The method 1200 may also include deriving a health score for the user (e.g., action 1204). The deriving of the health score for the user may be performable by example embodiments of the health score engine 350. The method 1200 may also include generating a first set of recommendations for the user based on the current set of information for the user (e.g., the final ranked list of recommendations, as described above and in the present disclosure) (e.g., action 1206). The method 1200 may also include generating, for each recommendation in the first set of recommendations, an achievable health score (e.g., action 1208). In an example embodiment, each achievable health score for each recommendation in the first set of recommendations may represent an update to the health score for the first user (as derived in action 1204) if the recommendation is selected by the user. For example, a user may have a health score derived in action 1204 to be a value of 80. A first recommendation in the first set of recommendations for the user (as generated in action 1206) may have an achievable health score of 81, a second recommendation in the first set of recommendations for the user (as generated in action 1206) may have an achievable health score of 80, a third recommendation in the first set of recommendations for the user (as generated in action 1206) may have an achievable health score of 85, a fourth recommendation in the first set of recommendations for the user (as generated in action 1206) may have an achievable health score of 82, and so on. In such an example, if the user selects the first recommendation, then the user's health score will or is expected to update to a score of 81. If, on the other hand, the user selects the second recommendation, then the user's health score does not or is not expected to change. If, on the other hand, the user selects the third recommendation, then the user's health score is expected to update to a score of 85. If, on the other hand, the user selects the fourth recommendation, then the user's health score is expected to update to a score of 82. And so on.

Example embodiments of the method 1200 of processing and/or managing health-related information may include or not include one or more of the actions described above and in the present disclosure, may include additional actions, operations, and/or functionality, may be performed in different sequences and/or combinations, and/or one or more of the actions, operations, and/or functionality may be combinable into a single action, operation, and/or functionality and/or divided into two or more actions, operations, and/or functionalities.

While various embodiments in accordance with the disclosed principles have been described above, it should be understood that they have been presented by way of example only, and are not limiting. Thus, the breadth and scope of the example embodiments described in the present disclosure should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

For example, “communication,” “communicate,” “connection,” “connect,” “call,” “calling,” or other similar terms should generally be construed broadly to mean a wired, wireless, and/or other form of, as applicable, connection between elements, devices, computing devices, telephones, processors, controllers, servers, networks, telephone networks, the cloud, and/or the like, which enable voice and/or data to be sent, transmitted, broadcasted, received, intercepted, acquired, and/or transferred (each as applicable).

Database (or memory or storage) may comprise any collection and/or arrangement of volatile and/or non-volatile components suitable for storing data. For example, memory may comprise random access memory (RANI) devices, read-only memory (ROM) devices, magnetic storage devices, optical storage devices, solid state devices, and/or any other suitable data storage devices. In particular embodiments, database may represent, in part, computer-readable storage media on which computer instructions and/or logic are encoded. Database may represent any number of memory components within, local to, and/or accessible by a processor and/or computing device.

Various terms used herein have special meanings within the present technical field. Whether a particular term should be construed as such a “term of art” depends on the context in which that term is used. Such terms are to be construed in light of the context in which they are used in the present disclosure and as one of ordinary skill in the art would understand those terms in the disclosed context. The above definitions are not exclusive of other meanings that might be imparted to those terms based on the disclosed context.

Words of comparison, measurement, and timing such as “at the time,” “equivalent,” “during,” “complete,” and the like should be understood to mean “substantially at the time,” “substantially equivalent,” “substantially during,” “substantially complete,” etc., where “substantially” means that such comparisons, measurements, and timings are practicable to accomplish the implicitly or expressly stated desired result.

Additionally, the section headings and topic headings herein are provided for consistency with the suggestions under various patent regulations and practice, or otherwise to provide organizational cues. These headings shall not limit or characterize the embodiments set out in any claims that may issue from this disclosure. Specifically, a description of a technology in the “Background” is not to be construed as an admission that technology is prior art to any embodiments in this disclosure. Furthermore, any reference in this disclosure to “invention” in the singular should not be used to argue that there is only a single point of novelty in this disclosure. Multiple inventions may be set forth according to the limitations of the claims issuing from this disclosure, and such claims accordingly define the invention(s), and their equivalents, that are protected thereby. In all instances, the scope of such claims shall be considered on their own merits in light of this disclosure, but should not be constrained by the headings herein.

What is claimed is:

1. A method of processing information, the method comprising:
establishing, by a processor, one or more communication channels;
receiving, via the one or more communication channels, a plurality of information for a first user; and
processing, by the processor, the received information for the first user, the processing including:
deriving a first ranked list for the first user, the deriving including:
selecting, from among the received information for the first user, one or more health-related information for the first user;
searching a recommendations database, the recommendations database being a database of available recommendations;
selecting, from the recommendations database, a plurality of health-related recommendations for the first user, the selected health-related recommendations being recommendations selected from a health perspective;
establishing a first score for each of the selected health-related recommendations for the first user based on the selected one or more health-related information for the first user; and
ranking the selected health-related recommendations for the first user to arrive at the first ranked list for the first user, the ranking of the selected health-related recommendations for the first user based on the first score of each of the selected health-related recommendations for the first user; and
deriving a second ranked list for the first user, the deriving including:
selecting, from among the received information for the first user, one or more financial-related information for the first user;
searching the recommendations database;
selecting, from the recommendations database, a plurality of financial-related recommendations for the first user, the selected financial-related recommendations being recommendations selected from a financial perspective;
establishing a second score for each of the selected financial-related recommendations for the first user based on the selected one or more financial-related information for the first user; and
ranking the selected financial-related recommendations for the first user to arrive at the second ranked list for the first user, the ranking of the selected financial-related recommendations for the first user based on the second score of each of the selected financial-related recommendations for the first user.

2. The method of claim 1, wherein the processing further includes:
deriving a third ranked list for the first user, the deriving including:
selecting, from among the received information for the first user, one or more historic accepted information for the first user, the historic accepted information being information pertaining to only those recommendations provided to the first user that were previously accepted by the first user;
searching the recommendations database;
selecting, from the recommendations database, a plurality of prediction-based recommendations for the first user, the selected prediction-based recommendations being recommendations selected based a likelihood of being selected by the first user, wherein the selecting of the prediction-based recommendations includes a consideration of the selected one or more historic accepted information of the user;
establishing a third score for each of the selected prediction-based recommendations for the first user based on a likelihood of being selected by the first user; and
ranking the selected prediction-based recommendations for the first user to arrive at the third ranked list for the first user, the ranking of the selected prediction-based recommendations for the first user based on the third score of each of the selected prediction-based recommendations for the first user.

3. The method of claim 2, wherein the processing further includes:
deriving a final ranked list for the first user, the deriving including:
processing, via a selection engine, the first ranked list, the second ranked list, and the third ranked list based on a predetermined criterion; and
ranking the results of the selection engine processing to arrive at the final ranked list for the first user; and
displaying, on a graphical display of the first user, the final ranked list of recommendations.

4. The method of claim 1, wherein the processing further includes:
deriving a final ranked list for the first user, the deriving including:
processing, via a selection engine, the first ranked list and the second ranked list based on a predetermined criterion; and
ranking the results of the selection engine processing to arrive at the final ranked list for the first user; and
displaying, on a graphical display of the first user, the final ranked list of recommendations.

5. The method of claim 1, wherein one or more of the following apply:
at least one of the communication channels are established with a computing device of the first user;
at least one of the communication channels are established with a computing device of an employer of the first user;
at least one of the communication channels are established with a computing device of an insurance company holding an insurance policy for the first user;
at least one of the communication channels are established with a computing device of a medical facility holding medical information for the first user.

6. The method of claim 1, wherein the selected one or more health-related information for the first user includes information pertaining to metabolic information of the first user.

7. The method of claim 1, wherein the selected one or more health-related information for the first user includes BMI, glucose, triglyceride, HDL cholesterol, and blood pressure information for the first user.

8. The method of claim 1, wherein the selected one or more health-related information for the first user includes health risk assessment (HRA) for the first user, number of steps by the first user, and images of the first user.

9. The method of claim 1, wherein the recommendations in the recommendations database includes recommendations on one or more products, medication, services, lifestyle changes, and/or fitness.

10. The method of claim 1, wherein the selected health-related recommendations are those recommendations that are most suitable for the first user based on the selected one or more health-related information of the first user.

11. The method of claim 1, wherein the selected financial-related recommendations are those recommendations that meet one or more of the following: most cost-effective from a standpoint of the first user, most cost-effective from a standpoint of an employer of the first user, most cost-effective from a standpoint of an insurance company holding an insurance premium of the first user, and/or most profitable from a standpoint of a medical provider for the first user.

12. The method of claim 1, further comprising:
establishing, by a processor, one or more other communication channels;
receiving, via one or more of the other communication channels, a plurality of information for a second user, the second user different from the first user; and
processing, by the processor, the received information for the second user, the processing including:
deriving a first ranked list for the second user, the deriving including:
selecting, from among the received information for the second user, one or more health-related information for the second user;
searching the recommendations database;
selecting, from the recommendations database, a plurality of health-related recommendations for the second user, the selected health-related recommendations being recommendations selected from a health perspective;
establishing a first score for each of the selected health-related recommendations for the second user based on the selected one or more health-related information for the second user; and
ranking the selected health-related recommendations for the second user to arrive at the first ranked list for the second user, the ranking of the selected health-related recommendations for the second user based on the first score of each of the selected health-related recommendations for the second user; and
deriving a second ranked list for the second user, the deriving including:
selecting, from among the received information for the second user, one or more financial-related information for the second user;
searching the recommendations database;
selecting, from the recommendations database, a plurality of financial-related recommendations for the second user, the selected financial-related recommendations being recommendations selected from a financial perspective;
establishing a second score for each of the selected financial-related recommendations for the second user based on the selected one or more financial-related information for the second user; and
ranking the selected financial-related recommendations for the second user to arrive at the second ranked list for the second user, the ranking of the selected financial-related recommendations for the second user based on the second score of each of the selected financial-related recommendations for the second user.

13. The method of claim 12, wherein, when the selected one or more health-related information for the first user is different from the selected one or more health-related information for the second user, the first ranked list for the first user is different from the first ranked list for the second user.

14. A method of processing healthcare-related information, the method comprising:
receiving, by a processor, a current set of information for a first user;
transforming, by the processor, the current set of information for the first user to a health score for the first user;
displaying, on a graphical display of the first user, the health score for the first user;
generating a first set of recommendations based on the current set of information for the first user;
displaying, on the graphical display of the first user, the first set of recommendations;
responsive to a selection, by the first user, of one of the recommendations from the first set of recommendations:
updating the current set of information for the first user based on the selected recommendation;
updating the health score for the first user based on the selected recommendation;
displaying, on the graphical display of the first user, the updated health score for the first user;
generating a second set of recommendations based on the updated current set of information for the first user; and
displaying, on the graphical display of the first user, the second set of recommendations.

15. The method of claim 14, wherein the current set of information for the first user includes a set of metabolic information for the first user and a set of lifestyle information for the first user.

16. The method of claim 15, wherein the set of metabolic information for the first user includes BMI, glucose, triglyceride, HDL cholesterol, and blood pressure information for the first user.

17. The method of claim 15, wherein the set of lifestyle information for the first user includes health risk assessment (HRA) for the first user, number of steps by the first user, and images of the first user.

18. The method of claim 15, wherein the transforming of the current set of information for the first user to the health score for the first user includes transforming, by the processor, the set of metabolic information for the first user to a metabolic score for the first user.

19. The method of claim 18, wherein the transforming of the current set of information for the first user to the health score for the first user includes transforming, by the processor, the set of lifestyle information for the first user to a lifestyle score for the first user.

20. The method of claim 18, wherein the metabolic score for the first user is between 50-70% of the health score for the first user.

21. The method of claim 19, wherein the lifestyle score for the first user is between 30-50% of the health score for the first user.

22. The method of claim 14, wherein the recommendations in the first set of recommendations includes recommendations on one or more products, medication, services, lifestyle changes, and/or fitness.

23. A method of processing healthcare-related information, the method comprising:
receiving, by a processor, a current set of information for a first user;
transforming, by the processor, the current set of information for the first user to a health score for the first user;
displaying, on a graphical display of the first user, the health score for the first user;
generating a first set of recommendations, including a first recommendation, based on the current set of information for the first user;
generating, for each recommendation in the first set of recommendations, an achievable health score, including a first achievable health score for the first recommendation, each achievable health score for each recommendation in the first set of recommendations representing an update to the health score for the first user if the recommendation is selected, wherein the first achievable health score represents the updated health score for the first user if the first recommendation is selected;
displaying, on the graphical display of the first user, the first set of recommendations, including the first recommendation; and
displaying, on the graphical display of the first user, the achievable health score for each recommendation in the first set of recommendations, including the first achievable health score for the first recommendation.

24. The method of claim 23, wherein the current set of information for the first user includes a set of metabolic information for the first user and a set of lifestyle information for the first user.

25. The method of claim 24, wherein the set of metabolic information for the first user includes BMI, glucose, triglyceride, HDL cholesterol, and blood pressure information for the first user.

26. The method of claim 24, wherein the set of lifestyle information for the first user includes health risk assessment (HRA) for the first user, number of steps by the first user, and images of the first user.

27. The method of claim 24, wherein the transforming of the current set of information for the first user to the health score for the first user includes transforming, by the processor, the set of metabolic information for the first user to a metabolic score for the first user.

28. The method of claim 27, wherein the transforming of the current set of information for the first user to the health score for the first user includes transforming, by the processor, the set of lifestyle information for the first user to a lifestyle score for the first user.

29. The method of claim 27, wherein the metabolic score for the first user is between 50-70% of the health score for the first user.

30. The method of claim 28, wherein the lifestyle score for the first user is between 30-50% of the health score for the first user.

31. The method of claim 23, further comprising:
responsive to a selection, by the first user, of one of the recommendations from the first set of recommendations:
updating the health score for the first user based on the selected recommendation;
displaying, on the graphical display of the first user, the updated health score for the first user;
updating the current set of information for the first user based on the selected recommendation;
generating a second set of recommendations, including a second recommendation, based on the updated current set of information for the first user;
generating, for each recommendation in the second set of recommendations, an achievable health score, including a second achievable health score for the second recommendation, each achievable health score for each recommendation in the second set of recommendations representing an update to the health score for the first user if the recommendation is selected, wherein the second achievable health score represents the health score for the first user if the second recommendation is selected;
displaying, on the graphical display of the first user, the second set of recommendations, including the second recommendation; and
displaying, on the graphical display of the first user, the achievable health score for each recommendation in the second set of recommendations, including the second achievable health score for the second recommendation.

32. The method of claim 23, wherein the recommendations in the first set of recommendations includes recommendations on one or more products, medication, services, lifestyle changes, and/or fitness.

33. A method of processing a medical claim, the method comprising:
establishing, by a processor, one or more communication channels;
receiving, via the one or more communication channels, a plurality of information for a first user;
processing, by the processor, the received information for the first user, the processing including:
deriving a first ranked list for the first user, the deriving including:
selecting, from among the received information for the first user, one or more health-related information for the first user;
searching a recommendations database, the recommendations database being a database of available recommendations;
selecting, from the recommendations database, a plurality of health-related recommendations for the first user, the selected health-related recommendations being recommendations selected from a health perspective;
establishing a first score for each of the selected health-related recommendations for the first user based on the selected one or more health-related information for the first user; and
ranking the selected health-related recommendations for the first user to arrive at the first ranked list for the first user, the ranking of the selected health-related recommendations for the first user based on the first score of each of the selected health-related recommendations for the first user;
deriving a second ranked list for the first user, the deriving including:
selecting, from among the received information for the first user, one or more financial-related information for the first user;
searching the recommendations database;
selecting, from the recommendations database, a plurality of financial-related recommendations for the first user, the selected financial-related recommendations being recommendations selected from a financial perspective;
establishing a second score for each of the selected financial-related recommendations for the first user based on the selected one or more financial-related information for the first user; and
ranking the selected financial-related recommendations for the first user to arrive at the second ranked list for the first user, the ranking of the selected financial-related recommendations for the first user based on the second score of each of the selected financial-related recommendations for the first user.
deriving a third ranked list for the first user, the deriving including:
selecting, from among the received information for the first user, one or more historic accepted information for the first user, the historic accepted information being information pertaining to only those recommendations provided to the first user that were previously accepted by the first user;
searching the recommendations database;
selecting, from the recommendations database, a plurality of prediction-based recommendations for the first user, the selected prediction-based recommendations being recommendations selected based a likelihood of being selected by the first user, wherein the selecting of the prediction-based recommendations includes a consideration of the selected one or more historic accepted information of the user;
establishing a third score for each of the selected prediction-based recommendations for the first user based on a likelihood of being selected by the first user; and
ranking the selected prediction-based recommendations for the first user to arrive at the third ranked list for the first user, the ranking of the selected prediction-based recommendations for the first user based on the third score of each of the selected prediction-based recommendations for the first user;
deriving a final ranked list for the first user, the deriving including:
processing, via a selection engine, the first ranked list, the second ranked list, and the third ranked list based on a predetermined criterion; and
ranking the results of the selection engine processing to arrive at the final ranked list for the first user; and
displaying, on a graphical display of the first user, the final ranked list of recommendations; and
responsive to a determination, by the processor, of a selection, by the first user, of a recommendation from among the final ranked list for the first user:
identifying, by the processor, a service provider associated with the first user's selected recommendation;
responsive to a determination, by the processor, that a claim has submitted for the first user's selected recommendation:
processing the claim for the first user's selected recommendation.

34. The method of claim 33, wherein the selected one or more health-related information for the first user includes information pertaining to metabolic information of the first user.

35. The method of claim 33, wherein the selected one or more health-related information for the first user includes BMI, glucose, triglyceride, HDL cholesterol, and blood pressure information for the first user.

36. The method of claim 33, wherein the selected one or more health-related information for the first user includes health risk assessment (HRA) for the first user, number of steps by the first user, and images of the first user.

37. The method of claim 33, wherein the recommendations in the recommendations database includes recommendations on one or more products, medication, services, lifestyle changes, and/or fitness.

38. The method of claim 33, wherein the selected health-related recommendations are those recommendations that are most suitable for the first user based on the selected one or more health-related information of the first user.

39. The method of claim 33, wherein the selected financial-related recommendations are those recommendations that meet one or more of the following: most cost-effective from a standpoint of the first user, most cost-effective from a standpoint of an employer of the first user, most cost-effective from a standpoint of an insurance company holding an insurance premium of the first user, and/or most profitable from a standpoint of a medical provider for the first user.
